(19) United States
(12) Patent Application Publication
(10) Pub. No.: US 2023/0060467 A1
(43) Pub. Date: Mar. 2, 2023

(54) SYSTEMS AND METHODS FOR IDENTIFYING ADAPTIVE IMMUNE CELL CLONOTYPES

(71) Applicant: 10x Genomics, Inc., Pleasanton, CA (US)

(72) Inventors: Wyatt James MCDONNELL, Pleasanton, CA (US); David Benjamin JAFFE, Pleasanton, CA (US); Sreenath KRISHNAN, Pleasanton, CA (US); Michael John Terry STUBBINGTON, Cambridge (GB)

(21) Appl. No.: 17/898,240

(22) Filed: Aug. 29, 2022

Related U.S. Application Data
(63) Continuation of application No. PCT/US2021/019120, filed on Feb. 22, 2021.
(60) Provisional application No. 62/983,485, filed on Feb. 28, 2020.
(60) Provisional application No. 63/011,783, filed on Apr. 17, 2020.

(51) Int. Cl. G16B 30/00; G16B 20/00

(52) U.S. Cl. CPC G16B 30/00; G16B 20/00

(57) ABSTRACT

A method for grouping immune cells within an immune cell receptor sequence dataset, is disclosed. An immune cell receptor sequence dataset is obtained from a sample. The dataset includes a plurality of full-length immune cell receptor sequences. Each full-length immune cell receptor sequence can comprise of at least one heavy chain region sequence and one light chain region sequence. Each immune cell receptor sequence is associated with an individual immune cell in the sample. The immune cell receptor sequences associated with a first immune cell and a second immune cell from the sample is compared using a comparison protocol. The first immune cell and the second immune cell are identified as members of the same clonotype if one or more immune cell receptor sequence comparison criteria is met.

### CROSS-REFERENCE TO RELATED APPLICATIONS

This application is a continuation of International Application No. PCT/US2021/019120, filed Feb. 22, 2021, which claims priority to and the benefit of U.S. Provisional Application No. 62/983,485, filed Feb. 28, 2020 and U.S. Provisional Application No. 63/011,783, filed Apr. 17, 2020. All applications are hereby incorporated by reference in their entirety as if fully set forth below and for all applicable purposes.

### FIELD

This description is generally directed towards systems and methods for identifying adaptive immune cell clonotypes using various single cell technologies. Single cell technologies within the disclosure can include single-modal (e.g., single cell immune cell receptor sequencing) and multi-modal (e.g., single cell immune cell receptor sequencing combined with, for example, gene expression, protein expression, and/or antigen capture technologies) platforms. Single cell sequencing technologies within the disclosure can include non-droplet and droplet-based microfluidic and array-based microwell and nanowell sequencing technologies. Specifically, systems and methods to identify adaptive immune cell clonotypes from sequence datasets derived from a multitude of biological settings, such as before and after vaccination or treatment with a drug or biological molecule, during chronic infection, and during immune-mediated hypersensitivity reactions, are disclosed herein. The identified adaptive immune cell clonotypes of the disclosure can be particularly important, for example, in the domains of antibody discovery, characterization, engineering, and more.

### BACKGROUND

The immune system recognizes and eliminates non-self threats through a complex and layered network of both innate and adaptive immune cells. Robust characterization of this response and discovery of novel cell types and antigen-specific populations has proven challenging to perform in a high-throughput fashion due to the limited number of analytes that can be measured simultaneously using flow cytometry, CyTOF, and similar assays. One approach to addressing these limitations is to utilize multi-modal single cell technologies, such as microfluidic droplet-based single cell techniques. Applications of these technologies include the analysis of pre- and post-vaccination T cells, B cells, and peripheral blood mononuclear cells from influenza vaccines or other vaccines (or of samples collected from individuals affected by diseases such as systemic lupus erythematosus and other autoimmune disorders, chronic viral infection, and acute/non-chronic viral infection), or T cells/B cells/PBMCs from individuals treated with a drug or biological molecule such as a checkpoint inhibitor, anti-cancer drug, monoclonal antibody, or antibody-drug conjugate. Importantly, these single cell assays allow users to learn the full and paired sequences of heterodimeric and extremely polymorphic immune cell receptors of adaptive lymphocytes, e.g., T cells and B cells, and to identify from which single cell (and its corresponding phenotype, genotype, and antigen specificity) a given immune receptor had originated. This relationship is masked or not directly observable using bulk DNA and RNA-based sequencing assays and is not captured in a cost-effective or high-throughput fashion in plate-based assays.

Using this framework, vaccine-specific T cell and B cell responses can be identified and used to implement an immune cell (B cells/T cells/PBMCs) clonotyping algorithm that resolves post-vaccination, post-disease or post-treatment activated immune cell antibody lineages at scale by combining untargeted and targeted gene expression, full-length immune cell receptor sequencing, surface protein expression and/or antigen capture, in addition to tag-based and genetic demultiplexing.

As such, there is a need for systems and methods that can utilize multi-modal single cell technologies to differentiate adaptive immune cell clonotypes with genomic sequencing, well beyond the setting of influenza vaccination. This is particularly important in the domains of antibody discovery, characterization, and antibody engineering, where assignment of the correct clonotypes is foundational to understanding how alterations in cell phenotype and antigen specificity are linked in immunotherapeutic products, passive and active vaccinations, and ease of engineering (presence or absence of glycosylation sites, addition or reversion of mutations in antibody lineages, etc.).

### SUMMARY

In one aspect, a method for grouping immune cells within an immune cell receptor sequence dataset, is disclosed. An immune cell receptor sequence dataset is obtained from a sample. In one aspect, the dataset includes a plurality of full-length immune cell receptor sequences each comprised of at least one heavy chain region sequence and one light chain region sequence. In some aspects, the dataset includes a plurality of full-length immune cell receptor sequences each comprised of a heavy chain region sequence and/or a light chain region sequence, a beta chain region sequence and/or an alpha chain region sequence, a gamma chain region sequence and/or a delta chain region sequence, or combinations thereof. Each immune cell receptor sequence is associated with an individual immune cell in the sample.

The immune cell receptor sequences associated with a first immune cell and a second immune cell from the sample is compared using a comparison protocol. The first immune cell and the second immune cell are identified as members of the same clonotype if one or more immune cell receptor sequence comparison criteria is met.

In another aspect, a system for grouping immune cells within an immune cell receptor sequence dataset, is disclosed. The system includes a data source and a processing unit. The data source is configured to obtain the immune cell receptor sequence dataset from a sample, the dataset including a plurality of full-length immune cell receptor sequences each comprised of at least one heavy chain region sequence and one light chain region sequence. In some aspects, the dataset includes a plurality of full-length immune cell receptor sequences each comprised of a heavy chain region sequence and/or a light chain region sequence, a beta chain region sequence and/or an alpha chain region sequence, a gamma chain region sequence and/or a delta chain region sequence, or combinations thereof. Each immune cell receptor sequence is associated with an individual immune cell in the sample. The processing unit is configured to receive the immune cell receptor sequence dataset from the data source. The processing unit hosts a comparison engine and an identification engine. The comparison engine is configured to compare the immune receptor sequences associated with a first immune cell and a second immune cell from the sample using a comparison protocol. The identification engine is configured to identify the first immune cell and the second immune cell as members of the same clonotype if one or more immune cell receptor sequence comparison criteria is met.

These and other aspects and implementations are discussed in detail herein. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 is a schematic illustration of a non-limiting example workflow for grouping immune cells within an immune cell receptor sequence dataset, in accordance with various embodiments.

FIG. 2 is a flow chart illustrating a non-limiting example method for grouping immune cells within an immune cell receptor sequence dataset, in accordance with various embodiments.

FIG. 3 is a diagram illustrating a non-limiting example system for grouping immune cells within an immune cell receptor sequence dataset, in accordance with various embodiments.

FIG. 4 is a block diagram that illustrates a computer system, upon which embodiments, or portions of the embodiments, may be implemented, in accordance with various embodiments.

FIG. 5 provides a schematic showing shared and non-shared mutations from the reference between two immune cells, in accordance with various embodiments.

FIG. 6 provides data outputs representing a single clonotype and associated various subclonotypes, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

It should be understood that any use of subheadings herein are for organizational purposes, and should not be read to limit the application of those sub headed features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, sub feature, and functionality of the specifically described feature.

Unless defined otherwise, all technical and scientific terms used herein have the same meaning as commonly understood by one of ordinary skill in the art to which their various embodiments belong.

All publications mentioned herein are incorporated herein by reference for the purpose of describing and disclosing devices, compositions, formulations and methodologies which are described in the publication and which might be used in connection with the present disclosure.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, unrecited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronical-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ, NEXTSEQ, and NOVASEQ Systems of Illumina, the DNBSEQ and BGISEQ platforms of Beijing Genomics Institute (BGI), the GRIDION and PROMETHION Systems of Oxford Nanopore Technologies, PACBIO SEQUEL Systems of Pacific Biosciences, and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.), which denotes a single or a grouping of genes (in DNA or RNA) that have undergone changes as referenced against a particular species or sub-populations within a particular species due to mutations, recombination/crossover or genetic drift.

In general, the methods and systems described herein accomplish sequencing of nucleic acid molecules including, but not limited to, DNA (e.g., genomic DNA), RNA (e.g., mRNA, including full-length mRNA transcripts, and small RNAs, such as miRNA, tRNA, and rRNA), and cDNA. In various embodiments, the methods and systems described herein accomplish genomic sequencing of nucleic acid molecules (e.g., DNA, RNA, and mRNA). In various embodiments, the methods and systems described herein accomplish genomic sequencing of immune cell receptor sequences (e.g., DNA, RNA, and mRNA). In various embodiments, the methods and systems described herein can accomplish transcriptome sequencing, e.g., whole transcriptome sequencing of mRNA encoding immune cell receptors. In some embodiments, the methods and systems described herein can also accomplish targeted genomic sequencing of nucleic acid molecules (e.g., DNA, RNA, and mRNA). In various embodiments, the methods and systems described herein accomplish single cell genomic sequencing, for example, single cell genomic sequencing of nucleic acid molecules (e.g., RNA and mRNA) encoding immune cell receptors of single cells, such as B cell receptors (BCRs) and T cell receptors (TCRs).

In various embodiments, the methods and systems described herein can include high-throughput sequencing technologies, e.g., high-throughput DNA and RNA sequencing technologies. In various embodiments, the methods and systems described herein can include high-throughput, higher accuracy short-read DNA and RNA sequencing technologies. In various embodiments, the methods and systems described herein can include long-read RNA sequencing, e.g., by sequencing cDNA transcripts in their entirety without assembly. In various embodiments, the methods and systems described herein can also, for example, segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long-range molecular context is not only preserved through a sequencing process, but is also preserved through the targeted enrichment process used in targeted sequencing approaches.

In general, the methods and systems described herein are directed to single cell analysis (including single- and multi-modal analyses) of genomic sequencing of nucleic acids (e.g., RNA and mRNA) encoding immune cell receptors of single cells, such as B cell receptors (BCRs) and T cell receptors (TCRs). Single cell analysis, including single cell multi-modal analyses (e.g., single cell immune cell receptor sequencing combined with, for example, gene expression, protein expression, and/or antigen capture technologies), as well as processing and sequencing of nucleic acids, in accordance with the methods and systems described in the present application are described in further detail, for example, in U.S. Pat. Nos. 9,689,024; 9,701,998; 10,011,872; 10,221,442; 10,337,061; 10,550,429; 10,273,541; and U.S. Pat. Pub. 20180105808, which are all herein incorporated by reference in their entirety for all purposes and in particular for all written description, figures and working examples directed to processing nucleic acids and sequencing and other characterizations of genomic material.

The term “B cells”, also known as B lymphocytes, refer to a type of white blood cell of the small lymphocyte subtype. They function in the humoral immunity component of the adaptive immune system by expressing and/or secreting antibodies. Additionally, B cells present antigens (they are also classified as professional antigen-presenting cells (APCs)) and secrete cytokines. In mammals, B cells mature in the bone marrow, which is at the core of most bones. In birds, B cells mature in the bursa of Fabricius, an immune organ where they were first discovered by Chang and Glick, (B for bursa) and not from bone marrow as commonly believed. B cells, unlike the other two classes of lymphocytes, T cells and natural killer cells, express B cell receptors (BCRs) on their cell membrane or secrete their BCRs if they have differentiated into long-lived plasma cells. BCRs allow a B cell to bind to specific antigens, against which it will initiate an antibody response.

The term “T cell”, also known as T lymphocytes, refer to a type of an adaptive immune cell. T cells develops in the thymus gland, hence the name T cell, and play a central role in the immune response of the body. T cells can be distinguished from other lymphocytes by the presence of a T cell receptor (TCR) on the cell surface. These immune cells originate as precursor cells, derived from bone marrow, and then develop into several distinct types of T cells once they have migrated to the thymus gland. T cell differentiation continues even after they have left the thymus. T cells include, but are not limited to, helper T cells, cytotoxic T cells, memory T cells, regulatory T cells, and killer T cells. Helper T cells stimulate B cells to make antibodies and help killer cells develop. Based on the T cell receptor chain, T cells can also include T cells that express αβ TCR chains, T cells that express γδ TCR chains, as well as unique TCR co-expressors (i.e., hybrid αβ-γδ T cells) that co-express the αβ and γδ TCR chains.

T cells can also include engineered T cells that can attack specific cancer cells. A patient's T cells can be collected and genetically engineered to produce chimeric antigen receptors (CAR). These engineered T cells are called CAR T cells, which forms the basis of the developing technology called CAR-T therapy. These engineered CAR T cells are grown by the billions in the laboratory and then infused into a patient's body, where the cells are designed to multiply and recognize the cancer cells that express the specific protein. This technology, also called adoptive cell transfer is emerging as a potential next-generation immunotherapy treatment.

T cells, such as the killer T cells can directly kill cells that have already been infected by a foreign invader. T cells can also use cytokines as messenger molecules to send chemical instructions to the rest of the immune system to ramp up its response. Activating T cells against cancer cells is the basis behind checkpoint inhibitors, a relatively new class of immunotherapy drugs that have recently been approved to treat lung cancer, melanoma, and other difficult cancers. Cancer cells often evade patrolling T cells by sending signals that make them seem harmless. Checkpoint inhibitors disrupt those signals and prompt the T cells to attack the cancer cells.

The term “naïve”, as used herein, can refer to B-lymphocytes or T-lymphocytes that have not yet reacted with an epitope of an antigen or that have a cellular phenotype consistent with that of a lymphocyte that has not yet responded to antigen-specific activation after clonal licensing.

The term “Fab”, also referred to as an antigen-binding fragment, refers to the variable portions of an antibody molecule with a paratope that enables the binding of a given epitope of a cognate antigen. The amino acid and nucleotide sequences of the Fab portion of antibody molecules are hypervariable. This is in contrast to the “Fc” or crystallizable fragment, which is relatively constant and encodes the isotype for a given antibody; this region can also confer additional functional capacity through processes such as antibody-dependent complement deposition, cellular cytotoxicity, cellular trogocytosis, and cellular phagocytosis.

The phrase “clonal selection” refers to the selection and activation of specific B lymphocytes and T lymphocytes by the binding of epitopes to B cell receptors or T cell receptors with a corresponding fit and the subsequent elimination (negative selection) or licensing for clonal expansion (positive selection) of a B or T lymphocyte after binding of an antigenic determinant.

The phrase “clonal expansion” refers to the proliferation of B lymphocytes and T lymphocytes activated by clonal selection in order to produce a clonal population of daughter cells with the same antigen specificity and functional capacity. In the case of T lymphocytes this antigen specificity is exact at the nucleotide and protein level and in the case of B lymphocytes this antigen specificity can be exact at the nucleotide and protein level or mutated relative to the parent population by mutations at the nucleotide level (and by extension the protein level). This enables the body to have sufficient numbers of antigen-specific lymphocytes to mount an effective immune response.

The term “cytokines” refers to a wide variety of intercellular regulatory proteins produced by many different cells in the body, which ultimately control every aspect of body defense. Cytokines activate and deactivate phagocytes and immune defense cells, enhance or inhibit the functions of the different immune defense cells, and promote or inhibit a variety of nonspecific body defenses.

The phrase “T helper lymphocytes”, also referred to as helper cells, refer to a type of white blood cell that orchestrate the immune response and enhance the activities of the killer T-cells (those that destroy pathogens) and B cells (antibody and immunoglobulin producers).

The phrase “affinity maturation” refers to the gradual modification of the paratope and entire B cell receptor as a result of somatic hypermutation. B lymphocytes with higher affinity B cell receptors that can 1) bind the epitope more tightly and 2) therefore bind the epitope for a longer period of time are able to proliferate more and survive longer. These B cells can eventually differentiate into plasma cells, which secrete their antibodies and form the basis of serum-mediated immunity.

The phrase “somatic hypermutation” (SHM) refers to a cellular mechanism by which the adaptive immune system adapts to foreign elements confronting it (e.g. viruses, bacteria, biomolecules). A major component of the process of affinity maturation, SHM diversifies B cell receptors used to recognize foreign elements (antigens) and allows the immune system to adapt its response to new threats during the lifetime of an organism. Somatic hypermutation involves a programmed process of mutation predominantly affecting select framework and complementarity-determining regions of immunoglobulin genes. Unlike germline mutation, SHM operates at the level of an organism's individual immune cells. These mutations are not transmitted to the organism's offspring, but are transmitted to daughter cells of individual B cell clones. Mistargeted somatic hypermutation is a likely mechanism in the development of B cell lymphomas and many other cancers. Somatic hypermutation can also lead to the acquisition of non-VDJ template DNA within B cell receptor sequences, such as LAIR1 insertions in malaria-specific neutralizing antibodies.

Somatic hypermutation is a distinct diversification mechanism from isotype switching (also called class switching). Mutations acquired during somatic hypermutation eventually lead to isotype switching, in which a B cell's antibody can be coupled to different functions by switching to a different Fc/constant region sequence. Isotype switching is an irreversible process, in that once a B cell has switched from a given constant region (e.g. IGHM) to a new constant region (e.g. IGHA1) it can no longer use the IgM constant region as the DNA encoding the IgM Fc is excised and removed during isotype switching.

The term “contig”, originating from the term “contiguous”, refers to a set of overlapping DNA segments that together represent a consensus region of DNA. In bottom-up sequencing projects, a contig refers to overlapping sequence data (reads); in top-down sequencing projects, contig refers to the overlapping clones that form a physical map of the genome that is used to guide sequencing and assembly. Contigs can thus refer both to overlapping DNA sequences and to overlapping physical segments (fragments) contained in clones depending on the context. Note that clone, in reference to overlapping clones, refers to individual bacteria or constructs (e.g. phagemids, cosmids, etc.) containing distinct insertions of genomes that were utilized in early efforts to map genomes

The phrase “heavy chain” refers to the large polypeptide subunit of an antibody (immunoglobulin). The first recombination event to occur is between one D and one J gene segment of the heavy chain locus. Any DNA between these two gene segments is deleted. This D-J recombination is followed by the joining of one V gene segment, from a region upstream of the newly formed DJ complex, forming a rearranged VDJ gene segment. All other gene segments between V and D segments are now deleted from the cell's genome. Primary transcript (unspliced RNA) is generated containing the VDJ region of the heavy chain and both the constant mu and delta chains (Cμ and Cδ) (i.e., the primary transcript contains the segments: V-D-J-Cμ-Cδ). The primary RNA is processed to add a polyadenylated (poly-A) tail after the Cμ chain and to remove sequence between the VDJ segment and this constant gene segment. Translation of this mRNA leads to the production of the IgM heavy chain protein and the IgD heavy chain protein (its splice variant). Expression of the immunoglobulin heavy chain with one or more surrogate light chains constitutes the pre-B cell receptor that allows a B cell to undergo selection and maturation.

The phrase “light chain” refers to the small polypeptide subunit of an antibody (immunoglobulin). The kappa (κ) and lambda (λ) chains of the immunoglobulin light chain loci rearrange in a very similar way, except that the light chains lack a D segment. In other words, the first step of recombination for the light chains involves the joining of the V and J chains to give a VJ complex before the addition of the constant chain gene during primary transcription. Translation of the spliced mRNA for either the kappa or lambda chains results in formation of the Ig κ or Ig λ light chain protein. Assembly of the Ig μ heavy chain and one of the light chains results in the formation of membrane bound form of the immunoglobulin IgM that is expressed on the surface of the immature B cell. B cells may express up to two heavy chains and/or two light chains in respectively rare and uncommon instances through a phenomenon known as allelic inclusion. This phenomenon can only be directly observed using single-cell technologies, though it can be inferred with a degree of uncertainty using a combination of bulk sequencing technologies and probabilistic inference via an extension of the birthday paradox.

The phrase “complementarity-determining regions” (CDRs) refers to part of the variable chains in immunoglobulins (antibodies) and T cell receptors, generated by B cells and T cells respectively, where these molecules are particularly hypervariable. The antigen-binding site of most antibodies and T cell receptors is typically distributed across these CDRs, collectively forming a paratope. However, there are many documented examples of paratopes that enable antigen recognition that fall outside of the CDRs. As the most variable parts of the molecules, CDRs are crucial to the diversity of antigen specificities and immune cell receptor sequences generated by lymphocytes.

V(D)J recombination is a genetic recombination mechanism that occurs in developing lymphocytes during the early stages of T and B cell maturation. Through somatic recombination, this mechanism produces a highly diverse repertoire of antibodies/immunoglobulins and T cell receptors (TCRs) found in B cells and T cells, respectively. This process is a defining feature of the adaptive immune system and these receptors are defining features of adaptive immune cells.

V(D)J recombination occurs in the primary immune organs (bone marrow for B cells and thymus for T cells) and in a generally random fashion. The process leads to the rearranging of variable (V), joining (J), and in some cases, diversity (D) gene segments. As discussed above, the heavy chain possesses numerous V, D, and J gene segments, while the light chain possesses only V and J gene segments. The process ultimately results in novel amino acid sequences in the antigen-binding regions of immunoglobulins and TCRs that allow for the recognition of antigens from nearly all pathogens including, for example, bacteria, viruses, and parasites. Furthermore, the recognition can also be allergic in nature or may recognize host tissues and lead to autoimmunity.

Human antibody molecules, including B cell receptors (BCRs), include both heavy and light chains, each of which contains both constant (C) and variable (V) regions, and are genetically encoded on three loci. The first is the immunoglobulin heavy locus on chromosome 14, containing the gene segments for the immunoglobulin heavy chain. The second is the immunoglobulin kappa (κ) locus on chromosome 2, containing the gene segments for part of the immunoglobulin light chain. The third is the immunoglobulin lambda (λ) locus on chromosome 22, containing the gene segments for the remainder of the immunoglobulin light chain.

Each heavy or light chain contains multiple copies of different types of gene segments for the variable regions of the antibody proteins. For example, the human immunoglobulin heavy chain region contains two C gene segments (Cμ and Cδ), 44 V gene segments, 27 D gene segments and 6 J gene segments. The number of given segments present in any individual can vary, as these gene segments are carried in haplotypes; for this reason, inference of both the alleles present within an individual and the germline sequence of those alleles is an important step in correctly identifying B cell clonotypes. The light chains possess two C gene segments (Cλ and Cκ) and numerous V and J gene segments, but do not have D gene segments. DNA rearrangement causes one copy of each type of gene segment to mate with any given lymphocyte, generating a substantial antibody repertoire. Approximately 10¹⁴ combinations are possible, with 1.5×10² to 3×10³ potentially removed via self-reactivity.

Accordingly, each naïve B cell makes an antibody with a unique Fab site through a series of gene recombinations, and later mutations, with the specific molecules of the given antibody attaching to the B cell's surface as a B cell receptor (BCR). These BCRs are then available to react with epitopes of an antigen.

When the immune system encounters an antigen, epitopes of that antigen will be presented to many B lymphocytes. B lymphocytes must first rearrange a heavy chain that enables pre-B cell receptor ligand binding. B lymphocytes that bind multivalent self-targets after rearrangement of the light chain too strongly are eliminated and die or undergo a secondary recombination event, while B cells that do not bind self-targets too strongly are licensed to exit the bone marrow. The latter becomes available to respond to non-self antigens and to undergo clonal expansion. This process is known as clonal selection.

Cytokines produced by activated CD4 T helper lymphocytes enable those activated B lymphocytes (B cells) to rapidly proliferate to produce large clones of thousands of identical B cells. More specifically, when under threat (i.e., via bacteria, virus, etc.), the body releases white blood cells by the immune system. CD4 T lymphocytes help the response to a threat by triggering the maturation of other types of white blood cell. They produce special proteins, called cytokines, have plural functions, including the ability to summon all of the other immune cells to the area, and also the ability to cause nearby cells to differentiate (become specialized) into mature B cells and T cells.

Accordingly, while only a few B cells in the body may have an antibody molecule that can bind a particular epitope, eventually many thousands of cells are produced with the right specificity, allowing the body's immune system to act en masse. This is referred to as clonal expansion. Natural phenomena such as IgA deficiency and murine transgenic models have shown that there are multiple paths by which a B cell receptor can acquire novel antigen specificity even from a very limited repertoire through the processes of somatic hypermutation and affinity maturation.

As the B cells proliferate, they undergo affinity maturation as a result of somatic hypermutation. This allows the B cells to “fine-tune” the paratopes of the antibody to more effectively fit with the recognized epitopes. B cells with high affinity B cell receptors on their surface bind epitopes more tightly and for a longer period of time, which enables these cells to selectively proliferate. Over the course of this proliferation and expansion, these variant B cells differentiate into plasma cells that synthesize and secrete vast quantities of antibodies with Fab sites that fit the target epitopes very precisely.

The phrase “immune cell” refers to a cell that is part of the immune system and that helps the body fight infections and other diseases. Immune cells include innate immune cells (such as basophils, dendritic cells, neutrophils, etc.) that are the first line of body's defense and are deployed to help attack the invading foreign cells (e.g., cancer cells) and pathogens. The innate immune cells can quickly respond to foreign cells and pathogens to fight infection, battle a virus, or defend the body against bacteria. Immune cells can also include adaptive immune cells (such as lymphocytes including B cells and T cells). The adaptive immune cells can come into action when an invading foreign cells or pathogens slip through the first line of body's defense mechanism. The adaptive immune cells can take longer to develop, because their behaviors evolve from learned experiences, but they can tend to live longer than innate immune cells. Adaptive immune cells remember foreign invaders after their first encounter and fight them off the next time they enter the body. Both types of immune cells employ important natural defenses in helping the body fight foreign cells and pathogens for fighting infections and other diseases.

Accordingly, the immune cells of the disclosure can include, but are not limited to, neutrophils, eosinophils, basophils, mast cells, monocytes, macrophages, dendritic cells, natural killer cells, and lymphocytes (such as B cells and T cells). The immune cells of the disclosure can further include dual expresser cells or DE (such as unique dual-receptor-expressing lymphocytes that co-express functional B cell receptor (BCR) and T cell receptor (TCR)), cells with adaptive immune receptors that may diversify or may not diversify (including immune cells expressing a chimeric antigen receptor with a fixed nucleotide sequence or with the capacity to mutate), and TCR co-expressors (i.e., hybrid αβ-γδ T cells) that co-express both αβ and γδ TCR chains.

The phrase “immune cell receptor”, “immune receptor”, or “immunologic receptor” refers to a receptor or immune cell receptor sequence, usually on a cell membrane, which can recognize components of pathogenic microorganisms (e.g., components of bacterial cell wall, bacterial flagella or viral nucleic acids) and foreign cells (e.g., cancer cells), which are foreign and not found naturally on the host cells, or binds to a target molecule (for example, a cytokine), and causes a response in the immune system. The immune cell receptors of the immune system can include, but are not limited to, pattern recognition receptors (PRRs), Toll-like receptors (TLRs), killer activated and killer inhibitor receptors (KARs and KIRs), complement receptors, Fc receptors, B cell receptors, and T cell receptors.

The phrase “immune cell receptor sequences” of an immune cell receptor include both heavy and light chains, each of which contains both constant (C) and variable (V) regions. For example, B cell receptors (BCRs) or B cell receptor sequences (including human antibody molecules) comprise of immunoglobulin heavy and light chains, each of which contains both constant (C) and variable (V) regions. Each heavy or light chain not only contains multiple copies of different types of gene segments for the variable regions of the antibody proteins, but also contains constant regions. For example, the BCR or human immunoglobulin heavy chain contains two (2) constant (Constant mu (Cμ) and delta (Cδ)) gene segments and forty four (44) Variable (V) gene segments, plus twenty seven (27) Diversity (D) gene segments, and six (6) Joining (J) gene segments. The BCR light chains also possess two (2) constant gene segments ((Constant lambda (Cλ) and kappa (Cκ) and numerous V and J gene segments, but do not have any D gene segments. DNA rearrangement (i.e., recombination events) in developing B cells can cause one copy of each type of gene segment to go in any given lymphocyte, generating an enormous antibody repertoire. Accordingly, the primary transcript (unspliced RNA) of a BCR heavy chain can be generated containing the VDJ region of the heavy chain and both the constant mu and delta chains (Cμ and Cδ), i.e., the heavy chain primary transcript can contain the segments: V-D-J-Cμ-Cδ). In case of the B cell receptor and human immunoglobulin light chain, the first step of recombination for the light chains involves the joining of the V and J chains to give a VJ complex before the addition of the constant chain gene during primary transcription. Translation of the spliced mRNA for either the constant κ (Cκ) or λ (Cλ) chains results in formation of the Ig κ or Iλ light chain protein.

In general, most T cell receptors (TCR) are composed of an alpha (α) chain and a beta (β) chain, each of which contains both constant (C) and variable (V) regions. Thus, the most common type of a T cell receptor is called an alpha-beta TCR because it is composed of two different chains, one α-chain and one beta β-chain. A less common type of TCR is the gamma-delta TCR, which contains a different set of chains, one gamma (γ) chain and one delta (δ) chain. The T cell receptor genes are similar to immunoglobulin genes for the BCR and undergo similar DNA rearrangement (i.e., recombination events) in developing T cells as for the B cells. For example, the alpha-beta TCR genes also contain multiple V, D, and J gene segments in their beta chains and V and J gene segments in their alpha chains, which are re-arranged during the development of the T cells to provide a cell with a unique T cell antigen receptor. Thus, the β-chain of the TCR can contain Vβ-Dβ-Jβ gene segments and constant domain (Cβ) genes resulting in a Vβ-Dβ-Jβ-Cβ sequence of the TCR β-chain. The re-arrangement of the alpha (α) chain of the TCR follows β chain rearrangement, and can include Vα-Jα gene segments and constant domain (Cα) genes resulting in a Vα-Jα-Cα sequence of the TCR α-chain. Similar to the alpha-beta TCRs, the TCR-γ chain is produced by V-J recombinations and can contain Vγ-Jγ gene segments and constant domain (Cγ) genes resulting in a Vγ-Jγ-Cγ sequence of the TCR γ-chain, while the TCR-δ chain is produced using V-D-J recombinations, and can contain Vδ-Dδ-Jδ gene segments and constant domain (Cδ) genes resulting in a Vδ-Dδ-Jδ-Cδ sequence of the TCR δ-chain.

The phrase “immune cell receptor constant region sequence” or “immune receptor constant region sequence” refers to the constant region or constant region sequence of an immune cell receptor. For example, the immune cell receptor constant region sequence or immune receptor constant region sequence can include, but is not limited to, the constant mu (Cμ) and delta (Cδ) region genes and sequences of a BCR and immunoglobulin heavy chain, the constant lambda (Cλ) and kappa (Cκ) region genes and sequences of a BCR and immunoglobulin light chain, the alpha constant (Cα) region genes and sequences of a TCR α-chain sequence, the beta constant (Cβ) region genes and sequences of a TCR β-chain sequence, the gamma constant (Cγ) region genes and sequences of a TCR γ-chain sequence, and the delta constant (Cδ) region genes and sequences of a TCR δ-chain sequence.

With this understanding of the immune cell's purpose in fighting off attacking foreign antigens, the pharmaceutical industry has strongly focused on designing vaccines with the ability to expand antibody lineages directed towards specific B cells with shared antigen specificity. To most effectively determine the efficacy of a vaccine or antitumor antibody therapy, it is essential to be able to accurately identify cell members of a clonotype, which potentially share common or similar BCRs or antigen specificity. The pharmaceutical industry has also directed its efforts to isolate antibodies and antibody lineages against non-foreign targets for the purpose of developing antibody-based therapeutics for a broad array of disease states including autoimmune disease (anti-inflammatory targets), cancer (checkpoint inhibitors and other targets), and other conditions such as osteoporosis. Similarly, knowing the fine specificities of different antibody lineages elicited by a vaccine is essential to understanding serum neutralization profiles and global epitope maps of an entire virus. This same concept applies to understanding how a patient's adaptive immune system can render drugs such as adalimumab ineffective through the emergence of anti-drug antibodies and distinct anti-drug antibody lineage.

To understand what constitutes members of a clonotype, one can start with the original progenitor cell for a given lineage of B cells, this progenitor cell commonly referred to as the parent clone, which is a single cell to which all daughter cells will be genetically related, though their B cell receptors and exact antigen specificity may differ and diverge over time. Collectively, this parent clone and all its daughter cells constitute a clonotype. As stated above, accurate identification of the members of a clonotype is critical not just from a biological perspective, but also from the biomedical perspective, as correct identification of all of the members of a given clonotype can be useful in the design of vaccines (e.g., which antibody lineages can be expanded by a vaccine or are expanded successfully or unsuccessfully by a vaccine), in the monitoring of B cell-mediated immune disease (e.g., myasthenia gravis, lupus, B cell lymphoma), and in other settings (what antibodies are found in the tumor microenvironment or other immune niches during clinical disease). Known approaches that attempt to group immune cell receptor sequences into groups with shared antigen specificity or members of the same clonotype include, but are not limited to: immcantation, Clonify, GLIPH, TCRdist, VDJTools, MiXCR, AbSolve, and the algorithms described in PMID: 23536288, PMID: 23898164, PMID: 25345460, etc. While some of these algorithms can successfully identify groups of T cells with shared antigen specificity using single-cell data (TCRdist, GLIPH), and the other algorithms use solely bulk receptor sequencing data (i.e., without access to heavy and light chain sequences), none of these algorithms attempt to approximate the true clonotypes for B cells while also attempting to mitigate for sources of noise in the data nor while using the additional specificity found in the antibody light chain. Antibody discovery efforts have shown that false-positive antibody candidates are more frequently found in randomly paired antibody libraries than in natively paired antibody libraries, demonstrating the importance of correct clonotype identification from both biological and pharmaceutical perspectives.

Therefore, in accordance with various embodiments, various methods are provided that use single-cell data to identify populations of single immune cells, including B cells and T cells, that are highly likely to have originated from the same parent clone.

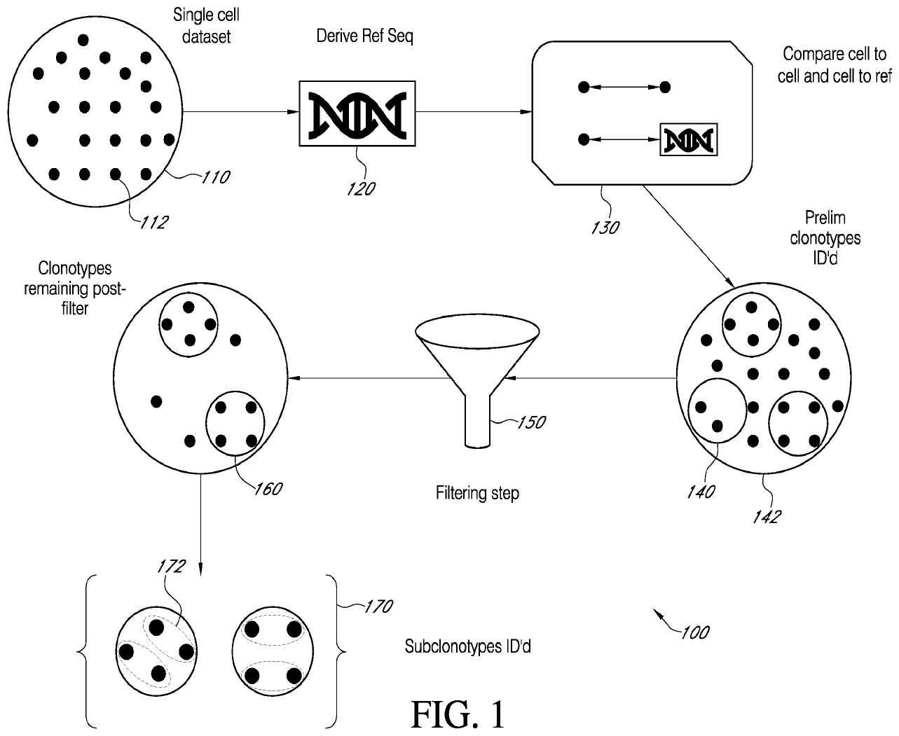

In accordance with various embodiments, a general schematic workflow is provided in FIG. 1 to illustrate a non-limiting example process for grouping immune cells within an immune cell receptor sequence dataset into clonotypes that best approximate cells sharing the same parental clone. The workflow can include various combinations of features, whether it be more or less features than that illustrated in FIG. 1. As such, FIG. 1 simply illustrates one example of a possible workflow.

FIG. 1 provides a schematic workflow 100, the workflow including an immune cell receptor sequence dataset 110 from a sample comprising a plurality of immune cells or from samples comprising a plurality of immune cells from one or more donors and from one or more timepoints or technical replicates or biological replicates 112. More detail regarding the acquisition of the particular dataset shown in 110 will be provided below. From that dataset, a reference immune receptor sequence 120 is identified. Reference immune receptor sequence 120 can be a donor reference sequence, universal reference sequence, or both. More detail regarding the acquisition of the reference immune receptor sequence 120, as well as further discussion related to the donor reference sequence and universal reference sequence will be provided below. All workflows can begin with a universal or user-supplied reference composed of the V, D, J, and C segments of the immune cell receptor loci, from which donor-specific reference sequences and their own deviations from the universal or user-supplied reference sequences are derived as described below.

With dataset 110 and reference sequence(s) 120 in hand, one or more comparisons 130 may be conducted. These comparisons can include comparing the immune receptor sequences associated with the immune cells of the dataset. Various cell to cell comparisons can be contemplated here and will be discussed in further detail below. These comparisons can also include comparing the immune receptor sequences of the various immune cells to the reference immune receptor sequence. Again, various reference to cell comparisons can be contemplated here and will be discussed in further detail below. It should be understood, and will be discussed below, that both comparisons are individually beneficial for grouping purposes, but can also be done together as part of the workflow. The presence of shared mutations across both highly dissimilar and similar single cells using the same variable gene for a given BCR or TCR chain may be taken into account in order to avoid falsely grouping cells into false-positive clonotypes.

Based on the one or more comparisons 130, one or more clonotypes 140 can be identified from dataset 110, as part of an identification protocol 142. Via identification protocol 142, the identification of clonotypes 140 is subject to meeting one or more comparison criteria. Detail regarding how comparisons 130, via the one or more comparison criteria, can lead to identification of the one or more clonotypes 140, will be provided below.

Identified clonotypes 140 can also be subject to one or more filters 150 that can function to remove specific cells from identified clonotypes, or eliminate whole clonotypes, that do not meet specific comparison criteria or are filtered out via the constraints imposed by the one or more filters 150. Detail regarding the filters will be provided below. Again, it should be understood that FIG. 1 simply illustrates a non-limiting example of the process for grouping immune cells. As such, the one or more filters 150 can activate after clonotypes are identified. Alternatively, the one or more filters can activate as part of identification protocol 142. Moreover, it is contemplated that one or more of filters 150 can activate before identification protocol 142. Even further, there need not be any active filters as part of the workflow 100.

Regardless of when or if one or more filters 150 are activated, an updated set of clonotypes 160 can be identified. As illustrated in FIG. 1, after application of filter(s) 150, two clonotypes 160 remained of the three originally identified clonotypes 140. It is understood, however, that in accordance with various embodiments, the one of more filters 150 need not be used, and that identification of the updated set of clonotypes 160 need not occur.

Regardless of when or if one or more filters 150 are activated, identified clonotype members can then be subcategorized into subclonotypes 172 as part of a subclonotype identification protocol 170. Detail related to subclonotypes and their identification will be provided below. Per the above, the one or more clonotypes 140 identified from dataset 110 as part of an identification protocol 142 can proceed directly to a subclonotype identification protocol 170. Alternatively, as illustrated in FIG. 1, clonotypes 160 remaining after activation of filters 150 can proceed to the subclonotype identification protocol 170. With identification of clonotypes and subclonotypes in hand, these results can then be output, as desired, for user review.

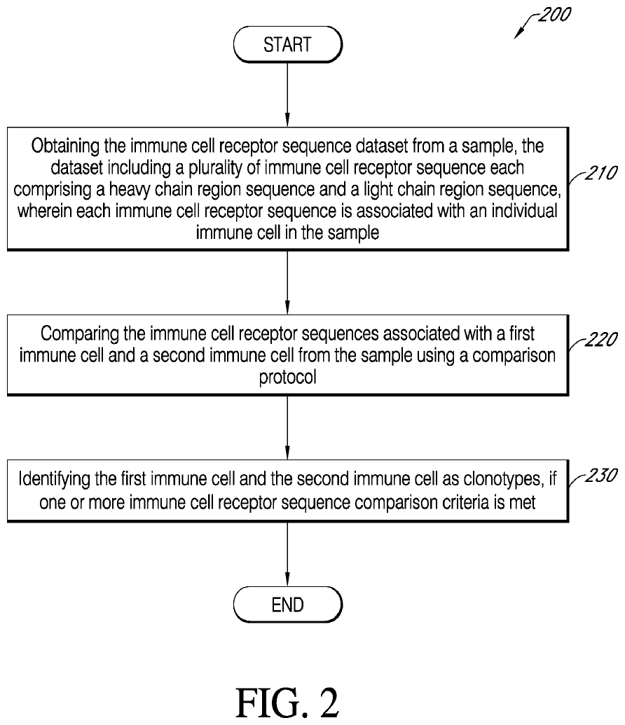

Referring now to FIG. 2, a flow chart is provided illustrating a method 200 for grouping immune cells represented within an immune cell immune receptor sequence dataset, in accordance with various embodiments. The method can comprise, at step 210, obtaining the immune cell receptor sequence dataset from a sample, the dataset including a plurality of immune cell receptor sequences each comprised of at least one heavy chain region sequence and/or one light chain region sequence, at least one beta chain region sequence and/or an alpha chain region sequence, at least one delta chain region sequence and/or a gamma chain region sequence, or combinations thereof, where each immune cell receptor sequence is associated with an individual immune cell in the sample. In various embodiments, the dataset can include a plurality of full-length immune cell receptor sequences. In various embodiments, the immune cell receptor sequences can further be comprised of immune cell receptor variable and/or constant region sequence(s). Details related to obtaining the dataset and the production of such a dataset is provided below.

The method can comprise, at step 220, comparing the immune cell receptor sequences associated with a first immune cell and a second immune cell from the sample using a comparison protocol. Various comparison protocols are contemplated and will be discussed in detail below. One or more comparison protocols can be utilized to sufficiently compare the appropriate immune cell receptor sequences. These protocols need not be utilized as a group or in any particular order. Each protocol can be independently capable of providing the necessary data as part of various method embodiments.

The method can comprise, at step 230, identifying the first immune cell and the second immune cell as members of the same clonotype, if one or more immune cell receptor sequence comparison criteria is met. Various comparison criteria are contemplated and will be discussed in detail below. One or more comparison criteria can be utilized to sufficiently identify clonotypes. These criteria need not be utilized as a group or in any particular order. Each criterion can be independently capable of providing the necessary data as part of various method embodiments.

In accordance with various embodiments, the comparison protocol can include receiving a reference immune cell receptor sequence, comparing the immune cell receptor sequences of the first immune cell and the second immune cell to the reference immune cell receptor sequence, and determining a number of shared mutations in the immune cell receptor sequences of the first immune cell and the second immune cell. The comparison criteria can be met when the immune cell receptor sequences of the first immune cell and the second immune cell share a pre-set number of mutations. In various embodiments, the pre-set number of shared mutations can be at least 25. In various embodiments, the pre-set number of shared mutations can be at least 10. In various embodiments, the pre-set number of shared mutations can be at least 11, at least 12, at least 13, at least 14, at least 15, at least 16, at least 17, at least 18 at least 19, at least 20, at least 21, at least 22, at least 23, or at least 24. It should be appreciated, however, that the optimal pre-set number of shared mutations can essentially be any number as it is dependent on the length of the immune cell receptor sequences being compared and can be optimized with expectation-maximization and fitting a mixture model as those with knowledge in the art would appreciate. The shared mutations can be point mutations, insertions, deletions, chromosomal mutations, or combinations thereof relative to the universal or user-supplied reference. The point mutation can be selected from the group consisting of substitution, insertion, and deletion, and the chromosomal mutation is selected from the group consisting inversion, deletion, duplication, translocation, and recombination. The point mutation can be a somatic hypermutation, defined as any of the point mutation categories.

In accordance with various embodiments, the comparison protocol can include calculating a probability value that the number of shared mutations occurred by chance. The protocol can further include determining that the comparison criteria has not being met if the probability value exceeds a pre-set probability threshold. In various embodiments, the probability value can be pre-set at between about 1% and 0.000001%. It should be understood, however, that the pre-set probability threshold can be calculated using Stirling numbers (an adaptive threshold) and is data dependent on multiple factors including, but not limited to: the number of cells in the dataset(s), the V genes not used in the dataset(s), etc.

In accordance with various embodiments, the comparison protocol can include comparing V and J segment portions of the immune cell receptor sequences associated with the first immune cell and the second immune cell. The comparison criteria can be met when the V and J segment portions have the same length. In accordance with various embodiments, the comparison protocol can include determining the number of base differences between the V and J segments portions associated with the first immune cell and the second immune cell, and determining that the comparison criteria is not met if the number of base differences exceeds a predetermined VJ base difference threshold. In various embodiments, the predetermined VJ base difference threshold can be between about 24 and about 50 bases. In various embodiments, the predetermined VJ base difference threshold is about 50 bases.

In accordance with various embodiments, the comparison protocol can include comparing only V segment portions of the immune cell receptor sequences associated with the first immune cell and the second immune cell. For example, the comparison criteria can be met when the V segment portions have the same length. In accordance with various embodiments, the comparison protocol can include determining the number of base differences between the V segment portions associated with the first immune cell and the second immune cell, and determining that the comparison criteria is not met if the number of differences exceed a predetermined V segment base difference threshold. In various embodiments, the predetermined V segment base difference threshold can be between about 24 and about 50 bases. In various embodiments, the predetermined V segment base difference threshold is about 50 bases.

In accordance with various embodiments, the comparison protocol can include comparing only the J segment portions of the immune cell receptor sequences associated with the first immune cell and the second immune cell. For example, the comparison criteria can be met when the J segment portions have the same length. In accordance with various embodiments, the comparison protocol can include determining the number of differences between the J segment portions associated with the first immune cell and the second immune cell, and determining that the comparison criteria is not met if the number of differences exceed a predetermined J segment base difference threshold. In various embodiments, the predetermined J segment base difference threshold can be between about 24 and 50 bases. In various embodiments, the predetermined J segment base difference threshold is about 50 bases.

In accordance with various embodiments, the comparison protocol can include comparing the D segment portions of the immune cell receptor sequences associated with the first immune cell and the second immune cell. For example, the comparison criteria can be met when the D segment portions have the same length. In accordance with various embodiments, the comparison protocol can include determining the number of differences between the D segment portions associated with the first immune cell and the second immune cell, and determining that the comparison criteria is not met if the number of differences exceed a predetermined D segment base difference threshold. In various embodiments, the predetermined D segment base difference threshold can be between about 24 and 50 bases. In various embodiments, the predetermined D segment base difference threshold is about 50 bases.

In accordance with various embodiments, comparison protocols can include comparing the lengths of the complementarity-determining regions (CDRs) associated with the first immune cell and the second immune cell. The comparison criteria can be met when the CDRs have the same length. In accordance with various embodiments, the comparison protocol can include determining the number of differences in the CDRs associated with the first immune cell and the second immune cell, and determining that the comparison criteria is not met when the number of differences exceeds a predetermined CDR threshold.

In accordance with various embodiments, the comparison protocol can include determining that the comparison criteria is not met if a first barcode associated with the first immune cell and a second barcode associated with the second immune cell are the same.

In accordance with various embodiments, the first immune cell and the second immune cell can be cell members of a two-cell clonotype, and methods can further comprise determining the number of CDR differences between the cell members, and determining that the comparison criteria is not met if the number of CDR differences exceed a determined two-cell threshold. In accordance with various embodiments, the two-cell threshold can have a value dependent on the number of shared mutations.

In accordance with various embodiments, the methods can further include identifying exact subclonotypes within the identified clonotype, wherein the exact subclonotype comprises a non-zero set of immune cells having identical V(D)J transcripts. The exact subclonotype can further comprise cells having characteristics selected from the group consisting of a same number of chains, an identical C segment, a same distance between a J stop codon and a C start codon, and combinations thereof. The exact subclonotype can comprise cells having two or three chains. The exact subclonotype can comprise cells with a shared inferred antigen specificity also shared or not shared by other subclonotypes. The exact subclonotype can comprise cells with shared gene expression, surface protein, intracellular protein, nucleotide variant, other cellular features, and combinations thereof.

In accordance with various embodiments, the first immune cell and the second immune cell can be both B cells. In accordance with various embodiments, the first immune cell and the second immune can be both “dual expressers,” which is are rare immune cells that express both a T cell receptor and a B cell receptor. In accordance with various embodiments, the first immune cell and the second immune cell can be both T cells.

In various embodiments, the reference immune receptor cell sequence can be a donor reference sequence, a universal reference sequence, or combinations thereof. In various embodiments, the reference immune cell receptor sequence can include each of the heavy and light chain V segments portions of the immune cell receptor sequences. In various embodiments, the donor reference immune cell receptor sequence can be derived for each of the heavy and light chain V segments portions by genotyping the V segments from the dataset. In various embodiments, the V segments portion can be a B cell heavy chain V segments portion, a B cell light chain V segments portion, a T cell alpha chain V segments portion, a T cell beta chain V segments portion, a T cell gamma chain V segments portion, a T cell delta chain V segments portion, or combinations thereof. In various embodiments, the donor reference sequence is derived from all the immune cells represented within the immune cell receptor sequence dataset, if there are sufficient immune cells present to determine a high-confidence estimate of the donor reference sequence.

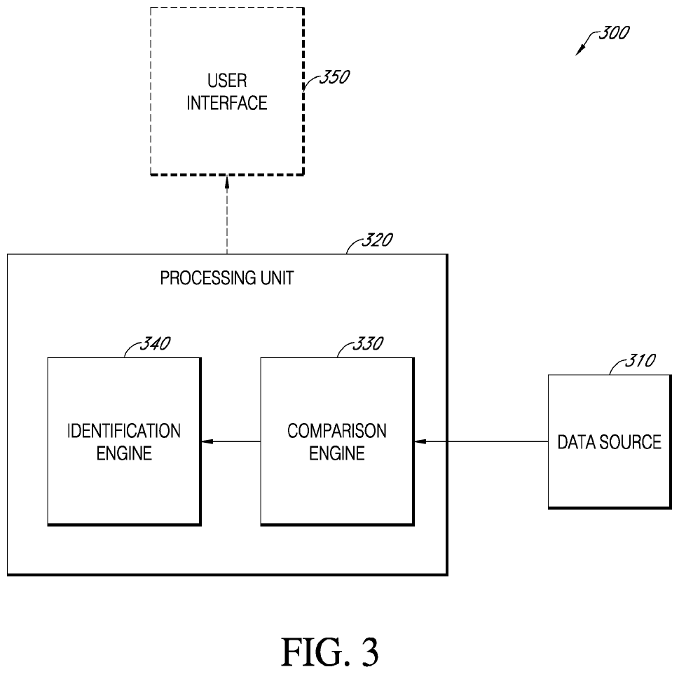

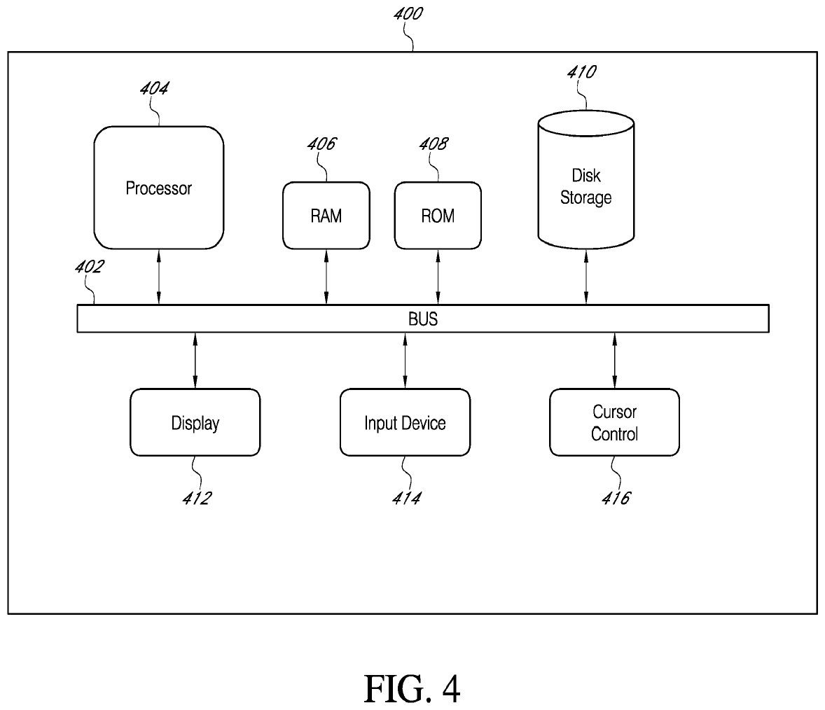

In accordance with various embodiments, FIG. 3 illustrates an example system 300 for grouping immune cells within an immune cell receptor sequence dataset. System 300 of FIG. 3 can include a data source 310, a processing unit 320, a user interface 350 (which can be optional in the provided example system 300 of FIG. 3. Processing unit 320 can include an identification engine 340 and a comparison engine 330. Data source 310 can be configured and arranged to obtain and/or store datasets for analysis by system 300. That dataset can be, for example, an immune cell receptor sequence dataset from a sample. The dataset obtained by and/or stored by data source 310 can be provided to processing unit 320. Processing unit 320 can be configured and arranged to receive the dataset (e.g., immune cell receptor sequence dataset) from data source 310. For example, the dataset can be provided to comparison engine 330 of processing unit 320, which can be configured and arranged to compare, for example, sequences (e.g., immune receptor sequences) associated with a first cell (e.g., first immune cell) and a second cell (e.g., second immune cell) from the sample using a comparison protocol. Output from comparison engine 320 can be provided to identification engine 330, which can be configured and arranged to identify the first immune cell and the second immune cell as members of the same clonotype if one or more immune cell receptor sequence comparison criteria is met. Interface 350 can be configured and arranged to receive output from processing unit 320 and display to a user as illustrated by FIG. 3. Interface 350 can also be configured and arranged to receive inputs from, for example, the user, with the inputs (e.g., instructions, parameters, etc.) associated with the analysis conducted by processing unit 320. See below for discussion of computer system 400, illustrated in FIG. 4, which can be implemented as part of the various system and method embodiments discussed herein.

It should also be understood that example system 300 simply shows one example of a system for grouping immune cells within an immune cell receptor sequence dataset. As such, the schematic illustration of system 300 on FIG. 3 is non-limiting as the location and interaction between the system components. For example, though system 300 shows engines 330 and 340 as part of unit 320, one or both engines 330/340 can be separated from unit 320. Further, data source 310 can be a component of the system 300, a separate component from system 300, or a sub-component on unit 320 or interface 350. As stated above, while FIG. 3 shows interface 350 receiving information, instructions or output from unit 320, it is well understood that interface can also be configured and arranged to deliver information and instructions to unit 320. Moreover, interface can be configured and arranged to communicate directly with data source 310.

In accordance with various embodiments, a system for grouping immune cells within an immune cell receptor sequence dataset, is provided. The system includes a data source and a processing unit. The data source is configured to obtain the immune cell receptor sequence dataset from a sample, the dataset including a plurality of full-length immune cell receptor sequences each comprised of at least one heavy chain region sequence and one light chain region sequence. In some aspects, the dataset includes a plurality of full-length immune cell receptor sequences each comprised of a heavy chain region sequence and/or a light chain region sequence, a beta chain region sequence and/or an alpha chain region sequence, a gamma chain region sequence and/or a delta chain region sequence, or combinations thereof. Each immune cell receptor sequence is associated with an individual immune cell in the sample. The processing unit is configured to receive the immune cell receptor sequence dataset from the data source. The processing unit hosts a comparison engine and an identification engine. The comparison engine is configured to compare the immune receptor sequences associated with a first immune cell and a second immune cell from the sample using a comparison protocol. The identification engine is configured to identify the first immune cell and the second immune cell as members of the same clonotype if one or more immune cell receptor sequence comparison criteria is met.

In accordance with various embodiments, the comparison engine can be configured to receive a reference immune cell receptor sequence, compare the immune cell receptor sequences of the first immune cell and the second immune cell to the reference immune receptor sequence, and determine a number of shared mutations in the immune cell receptor sequences of the first immune cell and the second immune cell.

In accordance with various embodiments, the identification engine can be further configured to determine that the comparison criteria is met when the immune cell receptor sequences of the first immune cell and the second immune cell share a pre-set number of mutations. The identification engine can be further configured to calculate a probability value that the number of shared mutations occurred by chance, and determine that the comparison criteria is not being met if the probability value exceeds a pre-set probability threshold. In accordance with various embodiments the pre-set probability threshold can be between about 1% and about 0.000001%.

In accordance with various embodiments, the pre-set number of shared mutations can be 25. The pre-set number of shared mutation is 10. The shared mutations can be, for example, point mutations, chromosomal mutations, or combinations thereof. The point mutation can be selected from the group consisting of substitution, insertion, and deletion, and the chromosomal mutation is selected from the group consisting inversion, deletion, duplication, translocation, and recombination. In various embodiments, the point mutation is a somatic hypermutation.

In accordance with various embodiments, the comparison engine can be further configured to compare V and J segments portions of the immune cell receptor sequences associated with the first immune cell and the second immune cell, and the identification engine can be further configured to determine that the comparison criteria is met when the V and J segments portions have the same length.

In accordance with various embodiments, the comparison engine can be further configured to determine the number of base differences between the V and J segments portions associated with the first immune cell and the second immune cell, and the identification engine can be further configured to determine that the comparison criteria is not met if the number of base differences exceed a predetermined VJ base difference threshold. In accordance with various embodiments, the predetermined VJ base difference threshold is between about 24 and about 50 bases. In accordance with various embodiments, the predetermined VJ base difference threshold is 50 bases.

In accordance with various embodiments, the comparison engine can be further configured to compare only V segments portions of the immune cell receptor sequences associated with the first immune cell and the second immune cell, and the identification engine can be further configured to determine that the comparison criteria is met when the V segments portions have the same length. The comparison engine can be configured to determine the number of base differences between the V segments portions associated with the first immune cell and the second immune cell, and the identification engine can be configured to determine that the comparison criteria is not met if the number of base differences exceed a predetermined V base difference threshold. The predetermined V base difference threshold can be between about 24 and about 50 bases. In accordance with various embodiments, the predetermined V base difference threshold is 50 bases.

In accordance with various embodiments, the comparison engine can be configured to compare only J segment portions of the immune cell receptor sequences associated with the first immune cell and the second immune cell, and the identification engine can be configured to determine that the comparison criteria is met when the J segment portions have the same length. The comparison engine can be configured to determine the number of base differences between the J segment portions associated with the first immune cell and the second immune cell, and the identification engine can be configured to determine that the comparison criteria is not met if the number of base differences exceed a predetermined J base difference threshold. In accordance with various embodiments, the predetermined J base difference threshold is between about 24 and about 50 bases. In accordance with various embodiments, the predetermined J base difference threshold is 50 bases.

In accordance with various embodiments, the comparison engine can be configured to compare the lengths of the complementarity-determining regions (CDRs) associated with the first immune cell and the second immune cell, and the identification engine can be further configured to determine that the comparison criteria is met when the CDRs have the same length. In accordance with various embodiments, the comparison engine can be configured to determine the number of differences in the CDRs associated with the first immune cell and the second immune cell, and the identification engine can be further configured to determine that the comparison criteria is not met when the number of differences exceeds a predetermined CDR threshold.

In accordance with various embodiments, the identification engine can be further configured to determine that the comparison criteria is not met if a first barcode associated with the first immune cell and a second barcode associated with the second immune cell are the same.

In accordance with various embodiments, the first immune cell and the second immune cell are cell members of a two-cell clonotype, wherein the comparison engine can be further configured to determine the number of CDR differences between the cell members, and the identification engine can be further configured to determine that the comparison criteria is not met if the number of CDR differences exceed a determined two-cell threshold. The two-cell threshold can have a value dependent on the number of shared mutations.

In accordance with various embodiments, the identification engine can be configured to identify exact subclonotypes within the identified clonotype, wherein the exact subclonotype comprises a non-zero set of immune cells having identical V, D, and J transcripts. The exact subclonotype can further comprise cells having characteristics selected from the group consisting of a same number of chains, an identical C segment, a same distance between a J stop codon and a C start codon, and combinations thereof. In accordance with various embodiments, the exact subclonotype comprises cells having two or three chains. In accordance with various embodiments, the exact subclonotype comprises cells having a shared inferred antigen specificity, wherein the shared inferred antigen specificity is also shared or not shared by other subclonotypes. In accordance with various embodiments, the exact subclonotype comprises cells having a shared gene expression, surface protein, intracellular protein, nucleotide variant, other cellular features, and combinations thereof.

In accordance with various embodiments, the first immune cell and the second immune cell are both B cells. In accordance with various embodiments, the first immune cell and the second immune cell are both T cells. In accordance with various embodiments, the first immune cell and the second immune cell are both dual-expresser (DE) cells, wherein the DE cells express both a T cell receptor and a B cell receptor. In accordance with various embodiments, the first immune cell and the second immune cell are both cells expressing a chimeric antigen receptor.

In accordance with various embodiments, the reference immune receptor cell sequence is a donor reference sequence, a universal reference sequence, and combinations thereof. In accordance with various embodiments, the donor reference sequence is derived from all the immune cells represented within the immune cell receptor sequence dataset, if there are sufficient immune cells present to determine a high-confidence estimate of the donor reference sequence. In accordance with various embodiments, the reference immune cell receptor sequence comprises each of the heavy and light chain V segments portions of the immune cell receptor sequences. The V segments portion of the immune cell receptor sequences can be a B cell heavy chain V segments portion, a B cell light chain V segments portion, a T cell alpha chain V segments portion, a T cell beta chain V segments portion, a T cell gamma chain V segments portion, a T cell delta chain V segments portion, and combinations thereof.

In accordance with various embodiments, the plurality of full-length immune cell receptor sequences comprise a variable region immune cell receptor sequence and a constant region immune cell receptor sequence. The variable region sequence can comprise a V through J segments portion of the immune cell receptor sequence.

In accordance with various embodiments, the identification engine is further configured to determine germline alleles for a donor V segments portion sequence. In accordance with various embodiments, the comparison engine is further configured to determine shared differences between immune cells within the immune cell receptor sequence dataset with disjoint CDR3 junction sequences and shared differences relative to a universal reference sequence, and the identification engine is further configured to determine that the identification of the shared differences are representative of germline mutations.

In accordance with various embodiments, the identification engine is further configured to join singletons to obtain a more frequent exact subclonotype if the sequences of the singletons are identical.

### Features of the Adaptive Immune Cell Clonotyping Workflow

Data Acquisition

In accordance with various embodiments, systems and methods within the disclosure include obtaining a dataset. That dataset can be a sequence dataset. The sequence dataset can be an immune cell receptor sequence dataset. The dataset can include a plurality of immune cell receptor sequences including both heavy chain region and light chain region sequences of antibodies and immunoglobulins, T-cell receptors (TCRs), or B-cell receptors (BCRs). The sequences in the dataset can represent the heavy chain variable region and light chain variable region sequences for each individual immune cell in a sample. The sequences in the dataset can represent the alpha, beta, gamma, and delta chains for each individual immune cell receptor in a dataset. In various embodiments, the immune cell receptor sequences can be comprised of immune cell receptor variable and/or constant region sequence(s).

The sequences in the dataset can represent the T cell receptors and B cell receptors for individual dual-expressing cells in the dataset. The immune cell can be a B cell or a T cell or a dual-expressing cell. Other examples of immune cell types that can be represented by the sequences in this dataset of the various embodiments herein, include, but are not limited to: cells with adaptive immune receptors that diversify or do not diversity (including cells expressed chimeric antigen receptors with fixed nucleotide sequences or with the capacity to mutate), TCR co-expressors (i.e., hybrid αβ-γδ T cells) that co-express the αβ and γδ TCR chains, cells with a diverse pool of chimeric antigen receptors, etc.

The variable regions of the heavy and light chains of the B cell immune receptors and the alpha/beta and gamma/delta chains of the T cell immune receptors of contain multiple copies of V and J gene segments, and in some instances D gene segments for the variable regions of the antibody and T cell receptor proteins. For example, the recombined heavy chain of the B cell immune receptors and beta and delta chains of the T cell receptors contains V, D, and J gene segments, whereas the recombined light chain of the B cell immune receptors and alpha and gamma chains of T cell receptors contains only V and J gene segments and lack a D gene segment. Accordingly, the immune cell receptor sequence dataset includes light, alpha, and gamma chain sequences containing the V and J segments and corresponding constant regions, and heavy, beta, and delta chain sequences containing the V, D, and J segments and corresponding constant regions.

The sample can be any biological sample, including for example, blood, tissue, cells, cell cultures, urine, or saliva. The biological sample may be derived from another sample. The sample may be a tissue sample, such as a biopsy, core biopsy, needle aspirate, or fine needle aspirate sample. The sample may be a fluid sample, such as a blood sample, urine sample, blister sample, or saliva sample. The sample may be a skin sample. The sample may be a cheek swab. The sample may be a plasma or serum sample. A sample can comprise of a fraction isolated from a bodily sample, which may be selected from the group consisting of blood, plasma, serum, urine, saliva, mucosal excretions, sputum, stool, and tears. Another example of a sample can be a tube of cells from a donor or subject, from a particular tissue at a particular point in time, and possibly enriched for particular cells. The terms donor and subject are used interchangeably herein. A donor or a subject is an individual from which samples are obtained. The donor or subject can be a mammalian or vertebrate subject, including for example, a human, swine, cow, camelid, lamprey, monkey, ape, dog, cat, mouse, or rat. The sample may be a cell or derivative of a cell (such as a cell nucleus). The sample may be a rare cell from a population of cells. The sample may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The sample may be a constituent of a cell. The sample may be or may include DNA, RNA, organelles, proteins, or any combination thereof. The sample may include or be processed to include a matrix (e.g., a gel or polymer matrix) comprising a cell or one or more constituents from a cell, such as DNA, RNA, organelles, proteins, or any combination thereof, from the cell. For a description of exemplary gel or polymer matrix embedded samples, see, e.g., U.S. Pat. Nos. 10,584,381; 10,428,326; and U.S. Pat. Pub. 20190100632, each of which are incorporated herein by reference in their entireties. The sample may be obtained from a tissue of a subject. The sample may be a hardened cell. Such hardened cell may or may not include a cell wall or cell membrane. In some instances, the sample may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix or cultured when comprising a gel or polymer matrix.

As discussed herein, various sequencing technologies can be used to obtain the immune cell receptor sequence data from the cells, e.g., the immune cells, in a sample. The sequencing technologies can include next generation sequencing (NGS) technology. As discussed within this disclosure, one example of the next generation sequencing technology is single cell sequencing technology. In various embodiments within the disclosure, such single cell sequencing technologies include, but are not limited to, non-droplet-based, droplet-based microfluidics, and array-based microwell- and nanowell-based technologies. Droplet-based microfluidic technologies (e.g., the 10× Genomics Chromium™ Single Cell Gene Expression Solution or the 10× Genomics Chromium™ Single Cell Immune Profiling Solution) generally utilize samples containing cells or nuclei of interest (e.g., an immune cell such as a B cell or a T cell, or an isolated nuclei from a B cell or T cell) and microfluidic chips to generate droplet emulsions that capture single cells from the sample and pairs them with uniquely barcoded beads, which are then used to generate barcoded cDNA libraries that are sequenced on a NGS platform, such as Illumina® sequencing instruments, to generate the sequencing output data. For a description of methods and systems suitable for such single cell and multi-modal analyses described herein, see e.g., U.S. Pat. Nos. 9,689,024; 9,701,998; 10,011,872; 10,221,442; 10,337,061; 10,550,429; 10,273,541; and U.S. Pat. Pub. 20180105808, which are all incorporated herein by reference in their entireties. Accordingly, as discussed herein, the various embodiments within the disclosure can include sequence dataset, e.g., immune cell receptor sequence dataset, from one or more biological samples, biological samples from one or more donors, and multiple libraries from one or more donors. It is understood that other sequencing technologies and platforms are also contemplated within the disclosure for generating the sequence output data from immune cell samples. Examples of these platforms include microwell and in situ based methods, droplet bioassay methods, plate-based methods, and similar.

The various embodiments, systems and methods within the disclosure further include processing and inputting the sequence output data, for example, the Chromium™ single-cell RNA-sequence output data discussed above. As an example, two compatible formats of the sequencing data can be as FASTQ files with headers in Illumina and non-Illumina format. One example of a software tool that processes and inputs the sequencing output data, e.g., the immune cell receptor sequence output data, for producing the immune cell receptor sequence dataset within the disclosure, can be the Cell Ranger™ Software. The Cell Ranger™ Software processes the Chromium single-cell RNA-sequence output data, e.g., the immune cell receptor sequence output data, and transforms such sequencing output data into input dataset, e.g., the immune cell receptor sequence output dataset, ready for analysis by the various embodiments, systems and methods within the disclosure. Accordingly, as an example within the disclosure, a sequence dataset, e.g., the immune cell receptor sequence dataset, can include all sequencing data obtained from a particular library type (e.g., TCR, BCR, cells with adaptive immune receptors that diversify or do not diversity, chimeric antigen receptors with fixed nucleotide sequences or with the capacity to mutate, TCR co-expressors, dual expresser or dual-expressing cells (a rare and unique type of lymphocyte expressing both a T cell receptor and a B cell receptor etc.), from one cell group, processed by running thorough the Cell Ranger™ Software pipeline. It is understood that other software tools are also contemplated within the disclosure for processing and transforming the sequencing output data into input files.

Identification and Handling of Indels

Various embodiments within the disclosure can recognize and display a single insertion or deletion in a contig relative to the reference sequence, so long as its length is divisible by three, is relatively short, and occurs within the V segment, not too close to its right end. These insertions and deletions, referred to as indels, could be germline, however most such events are already captured in an immune cell receptor reference sequence. In various embodiments, the full molecule can be annotated, i.e., indels and mutations found anywhere from the 5′ UTR through the sequenced portion of the constant region of the immune cell receptor can be identified by various embodiments within the disclosure. In various embodiments, the indels are identified only if the previous filtering criteria are met and the indels are compatible with a modulo 3 shift in the sequence with the indel relative to the other subclonotypes (i.e., only indels that result in a productive molecule are selected).

Reference Sequence Determination

In accordance with various embodiments, systems and methods within the disclosure can further include identifying a reference immune cell receptor sequence. The reference immune cell receptor sequence can be a donor reference immune cell receptor sequence (simply referred to as a donor reference sequence), universal reference immune cell receptor sequence (simply referred to as a universal reference sequence), or both.

In accordance with various embodiments within the disclosure, the donor reference sequences can be comprised of each of a heavy chain V segment and a light chain V segment portions of the immune cell receptor sequences. In accordance with various embodiments within the disclosure, the universal reference sequences can be comprised of each of a heavy chain V segment and a light chain V segment portions of the immune cell receptor sequences.

In accordance with various embodiments within the disclosure, the donor reference sequence can be derived from all the immune cells that are represented within the immune cell receptor sequence dataset, if there are sufficient immune cells present to determine a high-confidence estimate of the donor reference immune cell receptor sequence. In accordance with various embodiments within the disclosure, a V segment portions donor reference sequence (simply referred to as a V segment reference sequence) can be derived from all the immune cells that are represented within the immune cell receptor sequence dataset, if there are sufficient immune cells present to determine a high-confidence estimate of the reference immune cell receptor sequence. In accordance with various embodiments within the disclosure, if an insufficient number of immune cells is present to derive a donor reference sequence, such as a V segment reference sequence, then the analysis for clonotypes for that sequence (or a V segment in case of a V segment reference sequence), can default to the universal reference sequence. In accordance with various embodiments within the disclosure, the donor reference sequence can be calculated using Stirling numbers (probabilistic)-derived set of mutations, where all the immune cells with that donor reference sequence, for example, a V segment reference sequence, can be leveraged to identify a “null” distribution of mutations in the donor reference sequence relative to the universal reference sequence. In various embodiments, the donor reference sequence constitutes an individual's approximation of that germline sequence, for example, the germline V gene sequence.

In various embodiments, information about identification of clonotypes and subclonotypes determined by the comparison protocol comprising comparing the immune cell receptor sequences of the immune cell to the reference sequence (e.g., the donor reference sequence, universal reference sequence, or both), can be used, stored, and exported in accordance with various embodiments of the disclosure.

Accordingly, in various embodiments within the disclosure, the donor reference sequence can be derived for each of the heavy and light chain V segments portions by genotyping the V segments portion from the dataset. In various embodiments, the donor reference sequence is derived from all the immune cells represented within the immune cell receptor sequence dataset, if there are sufficient immune cells present to determine a high-confidence estimate of the donor reference sequence. In various embodiments, the V segments portion can be a B cell heavy chain V segments portion, a B cell light chain V segments portion, a T cell alpha chain V segments portion, a T cell beta chain V segments portion, a T cell gamma chain V segments portion, a T cell delta chain V segments portion, and combinations thereof. In accordance with various embodiments, the donor reference sequence can be derived for each of the alpha, beta, gamma, and delta V segments found in the immune cells from a donor or subject. The donor reference sequence, when derived for each of the heavy and light chain V segments portions by genotyping the V segments from the dataset, represent the V chains present in the donor's genome. The information related to the V segments is presumed to be imperfect because V segments vary in their expression frequency and due to the possibility of experimental and sampling bias therefore, large number of cells are required for the information to be complete. In other words, the more cells are present, the more complete the information will be with respect to the donor reference sequence for the heavy and light chain V segments. The second reason that the information related to the V segments is presumed to be imperfect is because it is difficult to accurately determine the last ˜15 bases in a V chain from transcript data. In accordance with various embodiments D segments may be derived using neural networks or hidden Markov chain models, though the D segment is similarly challenging to both align and derive due to non-templated nucleotide introduction and palindromic sequences that are part of the imprecise junctions formed during V(D)J recombination.

In accordance with various embodiments within the disclosure, the universal reference sequence can include a sequence found in a public database. The universal sequence can often be the single sequence for a given genomic segment that is found in the reference sequence for the given species. Accordingly, it can be presumed that a donor reference sequence is a modified version of the universal reference sequence that has mutations introduced, that are believed to have arisen in the germline sequence of the donor. As an example, the universal reference sequence can include J segment portions of the immune receptor sequences, D segment portions of the immune receptor sequences, or both. As an example, in accordance with various embodiments, the universal reference sequence, including for example any V, D, or J segments and constant regions, can be derived from whole genome sequencing, targeted genome sequencing, or derived from other bioassays using other computational approaches to produce a universal reference. In accordance with various embodiments, the universal reference sequence can also be derived for each of the alpha, beta, gamma, and delta V segments from whole genome sequencing, targeted genome sequencing, or derived from other bioassays using other computational approaches to produce a universal reference.

Cell Comparison and Grouping

In accordance with various embodiments, one or more comparison criteria can be utilized to sufficiently identify clonotypes or exact subclonotypes of cells, e.g., immune cells. These criteria need not be utilized as a group. It is understood that, certain criterion can be used independently or in combination with other steps discussed herein, while other criterion can only be used in combination with other steps discussed herein, in accordance with various embodiments within the disclosure.

Comparison Criteria: Same Length V and J Portion and Predetermined Threshold of Nucleotide Differences

In accordance with various embodiments, one comparison method may include comparing the V and J segments portions of the immune cell receptor sequences associated with any two immune cells from the sample. In such a comparison, the comparison criteria can be met if the V and J segments portions of the corresponding heavy and light chains of the two immune cells are of the same length. In such a comparison, the comparison criteria can be met if the V and J segments portions of the corresponding alpha, beta, gamma, and/or delta chains of the two immune cells are of the same length. It is understood that comparing the V and J segments portions step can be used independently or in combination with other steps discussed herein, in accordance with various embodiments within the disclosure.

However, the comparison criteria will still not be met and the immune cells will not be grouped as members of the same clonotype even if V and J segments portions of the corresponding heavy and light chains of the two immune cells are of the same length, if the number of base differences between the two given V and J segments portions between the two immune cells exceed a predetermined threshold. In one embodiment within the disclosure, the predetermined threshold base difference between the two given V and J segments portions combined can be up to 50 bases. In various embodiments, the predetermined threshold base difference between the two given V and J segments portions combined can be between about 25 and about 50 bases. In various embodiments, assuming an 850-bp long sequence, a predetermined threshold base difference of between about 85 and 100 bases can be set and still provide a reasonably high threshold that would tolerate a reasonable false positive rate.

Alternatively, the comparison can include comparing only the V segment portion or the J segment portion of the corresponding heavy and light chains of any two immune cells from the sample. In such a comparison, the comparison criteria can be met if either the V or the J segment portions of the corresponding heavy and light chains of the two immune cells are of the same length. In such a comparison within various embodiments within the disclosure, the comparison criteria can be met if either the V or the J segment portions of the corresponding alpha, beta, gamma, and/or delta chains of the two immune cells are of the same length. It is understood that comparing the V or J segment portion step can be used independently or in combination with other steps discussed herein, in accordance with various embodiments within the disclosure.

However, the comparison criteria will still not be met and the immune cells will not be grouped as members of the same clonotype even if the V or J segments portions of the corresponding heavy and light chains of the two immune cells are of the same length, if the number of differences between the two given V or J segments portions between the two immune cells exceed a predetermined threshold. In one embodiment within the disclosure, the predetermined threshold difference between either the two given V or J segments portions can be 50 bases. In various embodiments, the predetermined threshold difference between either the two given V or J segment portion can be between about 24 and about 50 bases.

Comparison Criteria: Same Length CDRs and Maximum Number of Nucleotide Differences

In accordance with various embodiments, systems and methods can further include comparing the lengths of the complementarity-determining regions (CDRs) associated with any two immune cells from the sample and grouping the cells as members of the same clonotype if one or more CDR comparison criteria is met. In such a comparison, the comparison criteria can be met when any one or more given CDRs of the corresponding heavy and/or light chains of the two immune cells have the same length. The CDR can be a CDR1, CDR2, CDR3, or any combination thereof. It is understood that comparing the lengths of the CDRs step can be used independently or in combination with other steps discussed herein, in accordance with various embodiments within the disclosure. In such a comparison, the comparison criteria can be met when the difference in length between any one or more given CDRs of the corresponding heavy and/or light chains of the two immune cells does not exceed a pre-set threshold. The threshold can be between about 1 and about 6. The total threshold and number of differences across all CDRs can produce sequences that are congruent modulo 3 for the comparison criteria to be met. The threshold can be weighted by inclusion of structural motifs.

In accordance with various embodiments, systems and methods can further include determining the number of differences in the corresponding CDRs associated with the two immune cells, and determining that the comparison criteria is not met when the number of differences exceeds a predetermined CDR threshold. An exemplary method for determining the number of differences in the CDR3 of the corresponding heavy and/or light chains of the two immune cells is as follows. Let N be “the number of DNA sequences that differ from the given CDR3 sequences by at most the number of observed differences.” More specifically, if cd is the number of differences between the given CDR3 nucleotide sequences, and n is the total length in nucleotides of the CDR3 sequences (for the two chains), we compute the total number N of strings of length n that are obtainable by perturbing a given string of length n, which is sum(choose(n,m), m=0 . . . =cd)). In various embodiments, determining the number of differences in the CDRs can result in a range of optimal values of mutations for CDRs of different lengths. Such values are data dependent and adaptive based on multiple factors. Alternatively, the method described above for determining the number of differences in CDR3 can be similarly employed for CDR1 and CDR2, as well for FWR1, FWR2, FWR3, and FWR4, and for the entire Fc region.

Comparison Criteria: Same Barcode

In accordance with various embodiments, systems and methods can further include determining that the comparison criteria for grouping any two immune cells as members of the same clonotype is not met if the barcodes associated with the two immune cells are the same. This is because, as discussed above, the Chromium™ single-cell RNA-sequencing technology, for example, takes samples containing cells, e.g., immune cell, or nuclei from cells of interest and uses microfluidic partitioning to capture single cells or nuclei to prepare uniquely barcoded, beads called Gel bead-in-EMulsions (GEMs), which are then used to derive barcoded cDNA libraries. As a result, all cDNAs from a single cell immune will have the same barcode, allowing the sequencing reads to be mapped back to their original single immune cell of origin. Accordingly, it can be presumed that immune cells with the same barcode are in fact of a single immune cell of origin.

Comparison Criteria: Comparison with the Reference and Shared Mutations

In accordance with various embodiments, systems and methods can further include comparing the immune cell receptor sequences of any two immune cell in the sample to the reference immune receptor sequence, and determining a number of shared mutations in the immune cell receptor sequences of the two immune cells. The two immune cells are both either B cells, T cells, dual expressers, cells with adaptive immune receptors that diversify or do not diversity (including cells expressed chimeric antigen receptors with fixed nucleotide sequences or with the capacity to mutate), TCR co-expressors (i.e., hybrid αβ-γδ T cells) that co-express the αβ and γδ TCR chains, and immune cells with a diverse pool of chimeric antigen receptors. The reference immune cell receptor sequence can be a donor reference sequence, universal reference sequence, or both, as discussed above. The comparison criteria can be met, and the two immune cells can be grouped as members of the same clonotype when the immune cell receptor sequences of the two immune cells share a pre-set number of mutations. The pre-set number of mutations can be at least 25. The pre-set number of mutations can be at least 10. In various embodiments, the pre-set number of shared mutations can be at least 11, at least 12, at least 13, at least 14, at least 15, at least 16, at least 17, at least 18 at least 19, at least 20, at least 21, at least 22, at least 23, or at least 24. It should be appreciated, however, that the optimal pre-set number of shared mutations can essentially be any number as it is dependent on the length of the immune cell receptor sequences being compared and can be optimized with expectation-maximization and fitting a mixture model as those with knowledge in the art would appreciate.

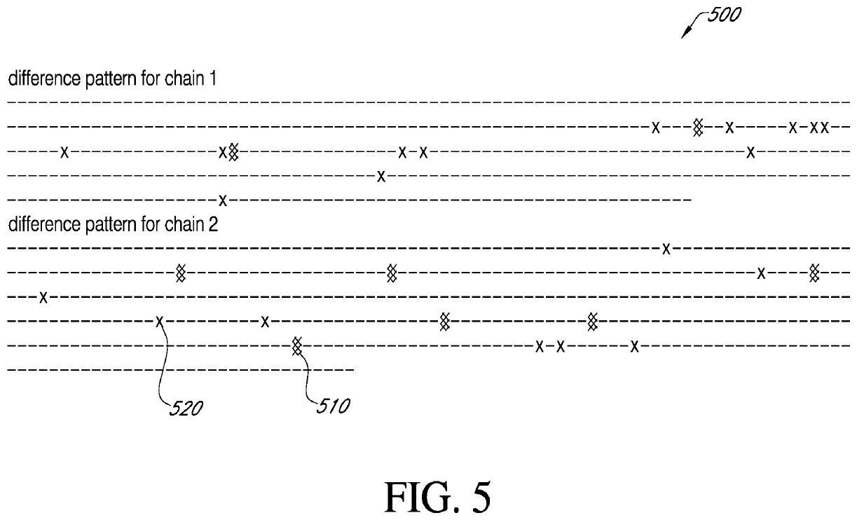

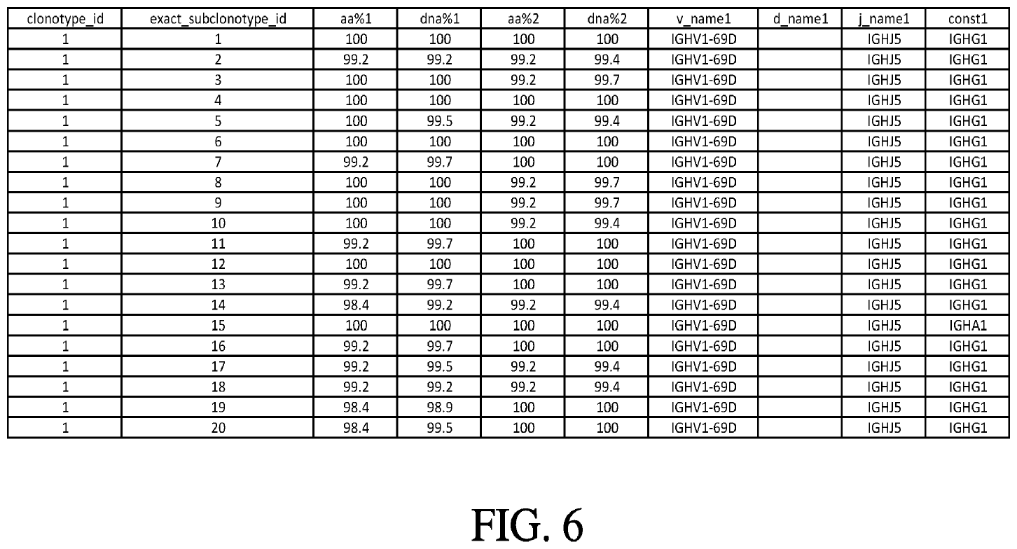

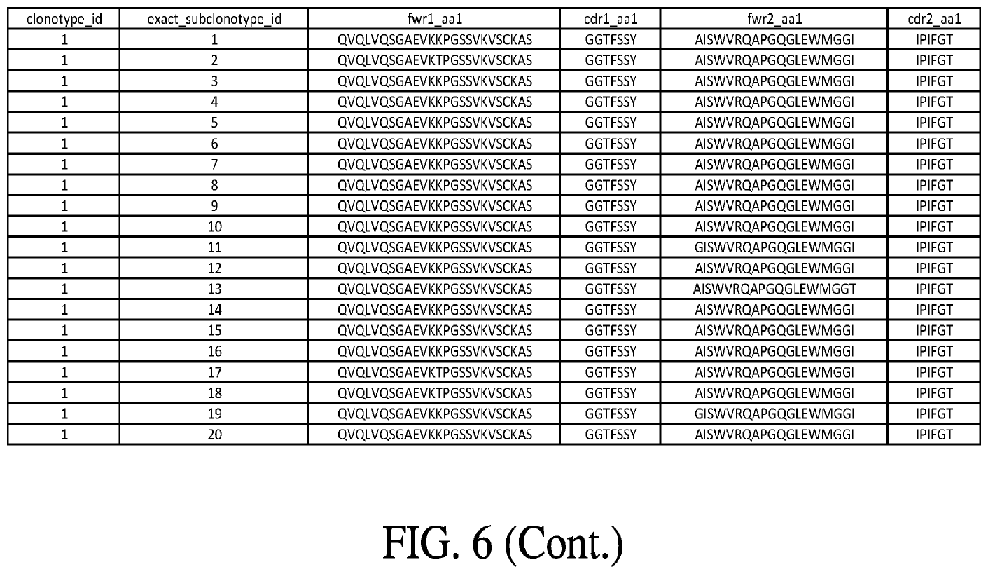

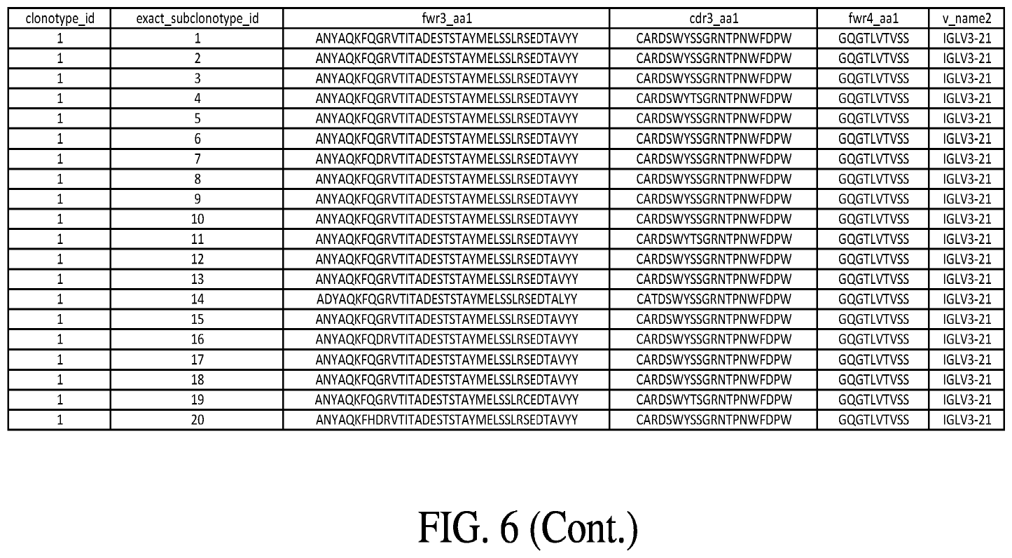

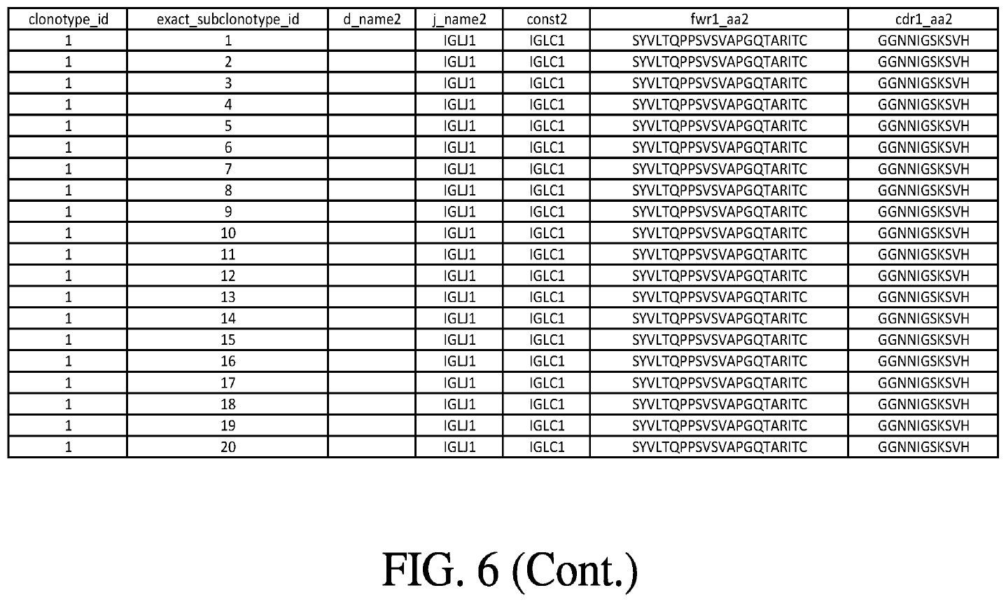

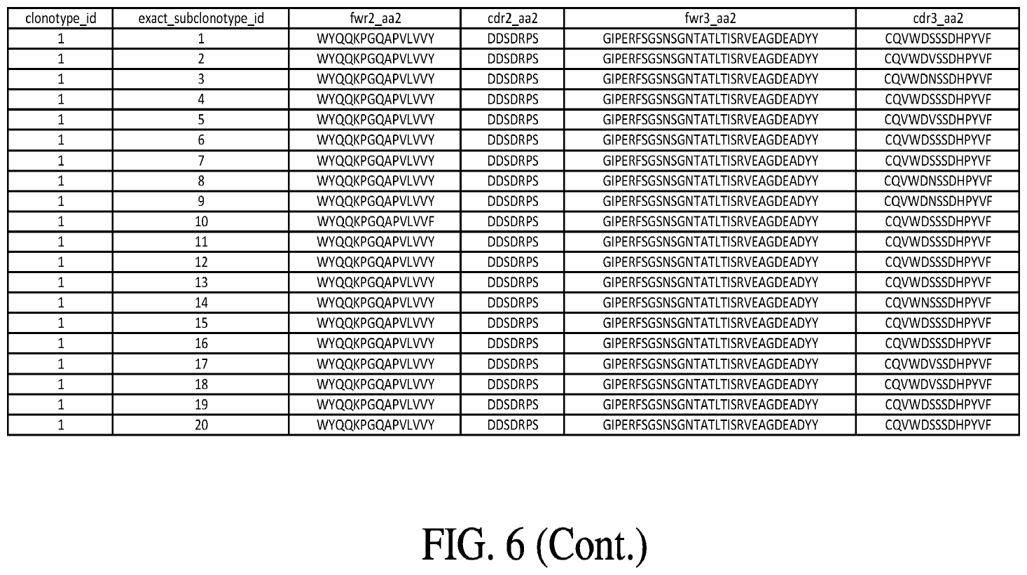

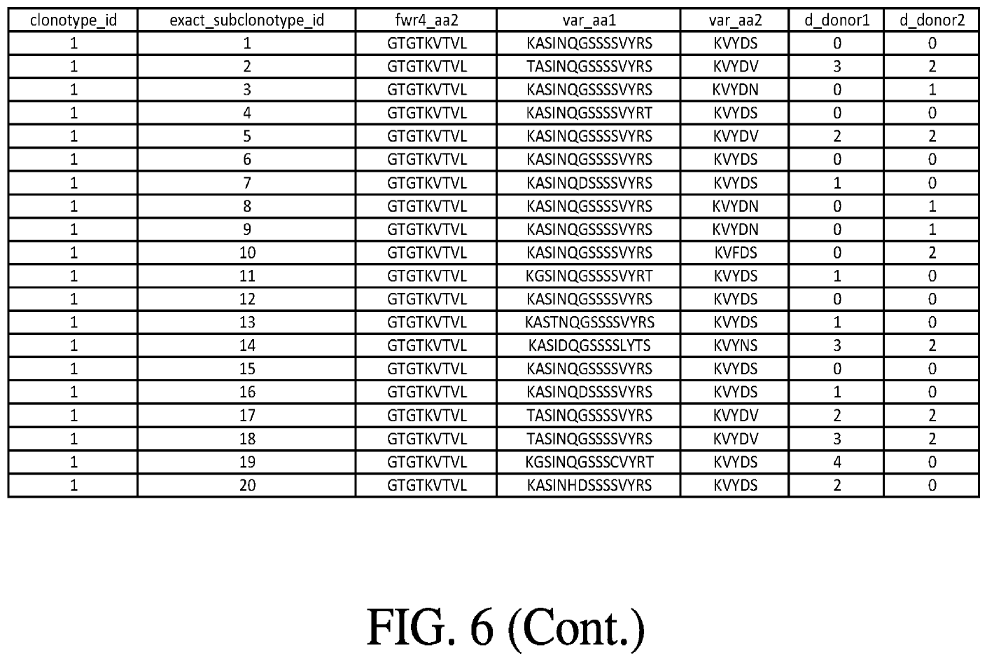

As an example, the methods within the disclosure determines shared mutations between two immune cells by determining common mutations from the reference sequence, using the donor reference for the V segments and the universal reference for the J segments. Shared mutations are presumed to be somatic hypermutations, which would be evidence of common ancestry. By using the donor reference sequences, most shared germline mutations are excluded, which can aid to the robustness of the various method and system embodiments within the disclosure. FIG. 5 provides a schematic 500 showing shared and non-shared mutations between two immune cells from the reference. Positions 510 represent the common mutations that the sequences of the two immune cells share from the reference sequence. Positions 520 represent difference from the reference sequence of any one of the immune cells. The schematic 500 shows comparing the sequences of any two immune cell in the sample to the reference sequence for determining the number of shared mutations in sequences of the two immune cells. FIG. 6 provides data outputs representing a single clonotype (represented by the “clonotype_id” column) and associated various subclonotypes (represented by the “exact_subclonotype_id” column), in accordance with various embodiments. Various other columns include features to help further understand and analyze each clonotype and subclonotype. These features can include, for example, aa % for amino acid percent identity with donor reference, outside junction region; dna % for nucleotide percent identity with donor reference; const1 for the constant region name; outside junction region; fwr*_aa for the framework region; d donor for the distance from the donor reference; var for positions in the specifically identified chain that vary across the clonotype; as well as numerous columns providing various framework region (fwr1, fwr2, fwr3, fwr4) and complementarity-determining regions (cdr1, cdr2, cdr3) information.

In accordance with various embodiments, systems and methods can further include calculating a probability value that the number of shared mutations occurred by chance. The methods can further include determining the comparison criteria as not being met if the probability value exceeds a pre-determined probability threshold. An exemplary method for calculating the probability value that the number of shared mutations occurred by chance is as follows. Given d shared mutations, and k total mutations (across the two immune cells), the probability value p that a sample with replacement of k items from a set whose size is the total number of bases in the V through J segments portions, yields at most k−d distinct elements. In various embodiments, the probability value can be pre-set at between about 1% and 0.000001%. It should be understood, however, that the pre-set probability threshold can be calculated using Stirling numbers (an adaptive threshold) and is data dependent on multiple factors including, but not limited to: the number of cells in the dataset(s), the V genes not used in the dataset(s), etc. Two immune cells sharing sufficiently many shared differences and sufficiently few CDR3 differences are deemed to be in the same clonotype. That is, the lower p is, and the lower N is, the more likely it is that the shared mutations represent bona fide shared ancestry. Accordingly, the smaller p*N is, the more likely it is that two immune cells lie in the same true clonotype. To group two immune cells into the same clonotype, the bound p*n≤C is satisfied, where C is the constant 1,000,000. This constant was arrived at by empirically balancing sensitivity and specificity across a large collection of datasets. In accordance with various embodiments, p can have a value dependent on the number of shared mutations.

Comparison Criteria for Two-Cell Clonotypes

In accordance with various embodiments, any two immune cells in the sample can be cell members of a two-cell clonotype. Methods within the disclosure include determining the number of CDR differences between the immune cell members and determining that the comparison criteria is not met if the number of CDR differences exceed a determined two-cell threshold. An additional comparison can be considered for determining a two-cell clonotype as follows: cd≤d/2 (where cd is the number of differences between the given CDR3 nucleotide sequences and d are shared mutations between the two cells). In accordance with various embodiments, the two-cell threshold therefore can have a value dependent on the number of shared mutations.

Comparison Criteria for Subclonotypes

In accordance with various embodiments, systems and methods can further include identifying subclonotypes within an identified clonotype. The subclonotype includes cells having identical V(D)J transcripts. The subclonotype can further include cells having an identical C segment, same distance between a J stop codon and a C start codon, or both. The subclonotype can include cells having two or three chains.

Comparison Criteria: Compute the Germline Alleles for the Donor's V Segments

In accordance with various embodiments, systems and methods can further include computing the germline alleles for the donor's V segments. Computing the germline alleles for the donor's V segments prevents spurious identification of false clonotypes containing “mutations” that are germline-derived and not true somatic hypermutations. The methods for deriving the donor and universal reference sequences are described above. A certain number of immune cells expressing the same V segment must be present in the dataset to derive a donor reference. In various embodiments, the number of immune cells can be a range or a pre-set threshold. Shared differences between immune cells with disjoint CDR3 junction sequences within the immune cell receptor sequence dataset and shared mutations relative to the universal reference are likely representative of germline mutations.

Comparison Criteria: Separately Joining Singletons

In accordance with various embodiments, systems and methods can further include filters for separately joining singletons. Singletons can have a level of UMI and read support comparable to that of the same chain (e.g., antibody immunoglobulin heavy and light chains and alpha and beta and gamma and delta chains of T cell receptors) and sequence found in immune cell barcodes with two or more chains. In various embodiments, for the purposes of visualization and reporting, the filter for joining singletons can be turned on (e.g., for excluding noisier data) or off (e.g., for reporting cleanest and most useful data). In various embodiments, singletons can be joined successfully to obtain a more frequent exact subclonotype if their nucleotide sequences are identical. In various embodiments, the ratio of the larger subclonotype to the smaller can be 10.

Comparison Criteria: Joining Clonotypes that Contain Indels within the CDR3 or Junction

Somatic hypermutation can give rise to one or more indels within the CDR3 sequence, whose length is divisible by three. These events are very rare, occurring at least five orders of magnitude less frequently than substitution mutations. As these events can create novel antigen specificity or enhance binding affinity, identification of clonotypes where this occurs is a biologically important problem to solve. In some embodiments, an additional filter can be used to identify these clonotypes. This filter, when activated, groups existing or preliminarily identified clonotypes by the length of the V segment stopping at the beginning of the CDR3. In some embodiments, this process can be accelerated while reducing computational cost by using either the heavy chain or the light chain. In some embodiments, both the heavy chain and light chain are used for this grouping procedure. For the first cell and second cell in the comparison, the number i of left-hand and right-hand bases of the sequence are counted; if i is greater than or equal to the length of the shorter of the sequences from the first cell or the second cell, the criteria for testing for an indel is met. In some embodiments, clonotypes can be grouped together if the additional clonotype comparison filters are met.

Noise Filtering

In accordance with various embodiments, systems and methods can further include data noise filters that, when activated, can provide the user more refined output.

An example of a filter is a cross-filter. If one specifies that two or more libraries arose from the same sample (i.e., from the same tube of immune cells), then the default behavior of the various embodiments herein, can be to “cross filter” so as to remove expanded exact subclonotypes that are present in one library but not another, in a fashion that would be highly improbable, assuming random draws of immune cells from the tube. Such observed behavior can be understood to arise when a plasma or plasma blast cell breaks up during or after pipetting from the tube, and the resulting fragments seed can yield ‘fake’ cells. This filter, presumably defaulted to being on during sample analysis of subclonotype identification, can also be turned off per user input. It is understood that the reverse is also contemplated.

Another example of a filter relates to a filter that, by default in various embodiments, removes exact subclonotypes that by their relationship to other exact subclonotypes, appear to arise from background mRNA or a phenotypically similar phenomenon. This filter, presumably defaulted to being on during sample analysis of subclonotype identification, can also be turned off per user input. It is understood that the reverse is also contemplated.

Another example of a filter relates to a filter that, by default in various embodiments, filters out exact subclonotypes having a base in V(D)J sequence that looks like it might be wrong. A Phred quality score (Q score) is a measure of the quality of the identification of the nucleobases generated by automated DNA sequencing. Various methods, in accordance with various embodiments herein, can find bases which are not Q60 for a barcode, not Q40 for two barcodes, are not supported by other exact subclonotypes, are variant within the clonotype, and which disagree with the donor reference. This filter, presumably defaulted to being on during sample analysis of subclonotype identification, can also be turned off per user input. It is understood that the reverse is also contemplated.

Another example of a filter relates to a filter that, by default in various embodiments, filters out chains from clonotypes that are weak and appear to be artifacts, perhaps arising from, for example, a stray mRNA molecule. This filter, presumably defaulted to being on during sample analysis of subclonotype identification, can also be turned off per user input. It is understood that the reverse is also contemplated.

Another example of a filter relates to a filter that, by default in various embodiments, identifies and filters out cells with low credibility, or barcode-associated rearrangements that artificially inflate the size of a given clonotype. This filter operates by using V(D)J sequence data in addition to one or more modes of data for the same cells. This filter is comprised of multiple steps, each of which can be run independently or in combinations with any of the other steps. These steps may include: (1) removal of V(D)J cells and chains that are not present in the second dataset (for example, remove of V(D)J cells if those cells are not also found in the orthogonal gene expression dataset); (2) for a clonotype of n cells, determining for each cell in the clonotype, the n nearest neighbors in an appropriate dimensional reduction or using a sensible distance metric to find these neighbors' gene expression or other dataset; and (3) calculating the credibility of a cell, where credibility is the percent of those nearest neighbors meeting at least one or more of the following criteria: (a) where the nearest neighbors are also V(D)J-called cells, (b) where the nearest neighbors are immune cells, e.g., B or T cells, identified by supervised analysis, (c) where the nearest neighbors are immune cells, e.g., B or T cells identified by supervised analysis, and (d) where the nearest neighbors are a non-B or non-T cell or a cell that should not otherwise express a B or T cell receptor. This filter can also use the nearest neighbor graph from various clustering algorithms (e.g. the Leiden or Louvain algorithms, and other commonly known algorithms) to calculate credibility of cells by: (1) measuring the geodesic distance between a cell and its n nearest neighbors in the graph; and (2) determining which of those nearest neighbors meet the comparison criteria listed above. This filter, presumably defaulted to being on for identifying and filtering out cells with low credibility, or barcode-associated rearrangements that artificially inflate the size of a given clonotype, can also be turned off per user input. It is understood that the reverse is also contemplated.

Another example of a filter relates to a filter that, by default in various embodiments, filters out onesie clonotypes (a clonotype or exact subclonotype having exactly one chain) having a single exact subclonotype, and that are light chain or TRA gene, and whose number of immune cells is less than, for example, 0.1% of the total number of immune cells. This filter, presumably defaulted to being on during sample analysis of subclonotype identification, can also be turned off per user input. It is understood that the reverse is also contemplated.

Another example of a filter relates to a filter that, by default in various embodiments, finds a foursie exact subclonotype that contains a twosie exact subclonotype having at least ten cells, it kills the foursie exact subclonotype, no matter how many immune cells it has. The foursies that are killed are believed to be rare odd artifacts arising from repeated cell doublets or, for example, GEMs (Gel bead-in-EMulsion) that contain two immune cells and multiple gel beads. This filter, presumably defaulted to being on during sample analysis of subclonotype identification, can also be turned off per user input. It is understood that the reverse is also contemplated.

Another example of a filter relates to a filter that, by default in various embodiments, filters out rare artifacts arising from contamination of oligos on gel beads. This filter, presumably defaulted to being on during sample analysis of subclonotype identification, can also be turned off per user input. It is understood that the reverse is also contemplated.

Another example of a filter relates to a filter that, by default in various embodiments, labels an exact subclonotype as improper if it does not have one chain of each type. This filtering option causes all improper exact subclonotypes to be retained, although they may be removed by other filters.

Another example of a filter relates to a filter that, by default in various embodiments, can be used to select exact subclonotypes within a specified range of generation probability, where the generation probability is calculated by calculating the likelihood of a specific rearrangement being generated relative to rearrangements generated in silico. In some embodiments, the generation probability is conditioned on the V gene used in the observed rearrangement. In some embodiments, spurious subclonotypes that may have been identified by de novo assembly or that arose due to chemistry errors can be removed by application of this filter in combination with other filters described. This filter, presumably defaulted to being on during sample analysis of exact subclonotype identification, can also be turned off per user input. It is understood that the reverse is also contemplated.

Yet another example of a filter relates to a filter that, by default in various embodiments, deletes any exact subclonotype having less than n chains. Such a filter can be used to “purify” a clonotype to display only exact subclonotypes having all their chains. Similarly, another example of a filtering option relates to a filter that, by default in various embodiments, deletes any exact subclonotype having less than n cells. Such a filter can be used for a very large and complex expanded clonotype, for which it may be desired to see a simplified view.

In accordance with various embodiments, systems and methods are also provided that are capable of exporting data files (e.g., FASTA, FASTQ, JSON, CSV, etc.) as part of a processing engine that contain the full-length sequences for each exact subclonotype of the various embodiments herein. In various embodiments, information relating to the full-length sequences for each exact subclonotype can be utilized for develop full-length recombinant antibodies and T cell receptors or chimeric molecules. In various embodiments, additional information can be exported alongside the full-length sequences or information for each exact subclonotype, such as cell type labels, antigen specificity scores and labels, credibility and other quality control statistics, and summary statistics such as number of features or molecules detected for a given assay modality.

In various embodiments, systems and methods are provided that are capable of exporting processed data to a visualization engine using information supplied to and calculated by the systems and methods of the various embodiments herein. In various embodiments, systems and methods are also provided that are capable of exporting both machine-readable and human-readable processed data in a variety of appropriate formats. In various embodiments, systems and methods are also provided that are capable of providing annotations and metadata for sets of barcodes or sets of immune cells to be included in data export or analysis within various embodiments herein.

### Computer System

FIG. 4 is a block diagram that illustrates a computer system 400, upon which embodiments of the present teachings may be implemented. For example, exemplary system 300, illustrated in FIG. 3 and discussed above, can include or implement computer system 400 to execute the analyses of system 300 and the various embodiments provided.

In various embodiments of the present teachings, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT) or liquid crystal display (LCD), for displaying information to a computer user. An input device 414, including alphanumeric and other keys, can be coupled to bus 402 for communicating information and command selections to processor 404. Another type of user input device is a cursor control 416, such as a mouse, a trackball or cursor direction keys for communicating direction information and command selections to processor 404 and for controlling cursor movement on display 412. This input device 414 typically has two degrees of freedom in two axes, a first axis (i.e., x) and a second axis (i.e., y), that allows the device to specify positions in a plane. However, it should be understood that input devices 414 allowing for 3-dimensional (x, y and z) cursor movement are also contemplated herein.

Consistent with certain implementations of the present teachings, results can be provided by computer system 400 in response to processor 404 executing one or more sequences of one or more instructions contained in memory 406. Such instructions can be read into memory 406 from another computer-readable medium or computer-readable storage medium, such as storage device 410. Execution of the sequences of instructions contained in memory 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software. The term “computer-readable medium” (e.g., data store, data storage, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as memory 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, Rust, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400 of Appendix D, whereby processor 404 would execute the analyses and determinations provided by these engines, subject to instructions provided by any one of, or a combination of, memory components 406/408/410 and user input provided via input device 414.

Digital Processing Device

In various embodiments, the systems and methods described herein can include a digital processing device or use of the same. In various embodiments, the digital processing device can include one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In some embodiments, the non-volatile memory comprises dynamic random-access memory (DRAM). In various embodiments, the non-volatile memory comprises ferroelectric random-access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing-based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

Computer Program

In various embodiments, the systems and methods disclosed herein can include at least one computer program or use at least one computer program. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® .NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or eXtensible Markup Language (XML). In various embodiments, a web application is written to some extent in a presentation definition language such as Cascading Style Sheets (CSS). In various embodiments, a web application is written to some extent in a client-side scripting language such as Asynchronous Javascript and XML (AJAX), Flash® Actionscript, Javascript, or Silverlight®. In various embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C#, Objective-C, Java™ Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelera-tor®, Celsius, Bedrock, Flash Lite, .NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo DSi Shop.

Standalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Rust, Objective-C, COBOL, Delphi, Eiffel, Java™, Lisp, Python™, Visual Basic, and VB.NET, or combinations thereof. Compilation is often performed, at least in part, to create an executable program. In various embodiments, a computer program includes one or more executable complied applications.

Web Browser Plug-In

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, Rust, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire-fox®, Google® Chrome, Apple® Safari®, Opera Software® Opera®, and KDE Konqueror. In various embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object oriented databases, object databases, entity-relation-ship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

### RECITATION OF EMBODIMENTS

Embodiment 1: A method for grouping immune cells within an immune cell receptor sequence dataset, the method comprising: obtaining the immune cell receptor sequence dataset from a sample, the dataset including a plurality of full-length immune cell receptor sequences each comprised of at least one heavy chain region sequence and one light chain region sequence, wherein each immune cell receptor sequence is associated with an individual immune cell in the sample; comparing the immune cell receptor sequences associated with a first immune cell and a second immune cell from the sample using a comparison protocol; and identifying the first immune cell and the second immune cell as members of the same clonotype, if one or more immune cell receptor sequence comparison criteria is met.

Embodiment 2: The method of embodiment 1, the comparison protocol including: receiving a reference immune cell receptor sequence; comparing the immune cell receptor sequences of the first immune cell and the second immune cell to the reference immune cell receptor sequence; and determining a number of shared mutations in the immune cell receptor sequences of the first immune cell and the second immune cell.

Embodiment 3: The method of embodiment 1 or 2, wherein the comparison criteria is met when the immune cell receptor sequences of the first immune cell and the second immune cell share a pre-set number of mutations.

Embodiment 4: The method of any of embodiments 1-3, the comparison protocol including: calculating a probability value that the number of shared mutations occurred by chance; and determining the comparison criteria as not being met if the probability value exceeds a pre-set probability threshold.

Embodiment 5: The method of any of embodiments 1-4, wherein the pre-set probability threshold is between about 1% and about 0.000001%.

Embodiment 6: The method of any of embodiments 1-5, wherein the pre-set number of shared mutations is 25.

Embodiment 7: The method of any of embodiments 1-6, wherein the pre-set number of shared mutation is 10.

Embodiment 8: The method of any of embodiments 1-7, wherein the shared mutations can be point mutations, chromosomal mutations, or combinations thereof.

Embodiment 9: The method of any of embodiments 1-8, wherein the point mutation is selected from the group consisting of substitution, insertion, and deletion, and the chromosomal mutation is selected from the group consisting inversion, deletion, duplication, translocation, and recombination.

Embodiment 10: The method of any of embodiments 1-9, wherein the point mutation is a somatic hypermutation.

Embodiment 11: The method of any of embodiments 1-10, the comparison protocol comprising comparing V and J segments portions of the immune cell receptor sequences associated with the first immune cell and the second immune cell, wherein the comparison criteria is met when the V and J segments portions have the same length.

Embodiment 12: The method of any of embodiments 1-11, further including determining a number of base differences between the V and J segments portions associated with the first immune cell and the second immune cell, and determining that the comparison criteria is not met if the number of base differences exceed a predetermined VJ base difference threshold.

Embodiment 13: The method of any of embodiments 1-12, wherein the predetermined VJ base difference threshold is between about 24 and about 50 bases.

Embodiment 14: The method of any of embodiments 1-13, wherein the predetermined VJ base difference threshold is 50 bases.

Embodiment 15: The method of any of embodiments 1-10, the comparison protocol comprising comparing only V segments portions of the immune cell receptor sequences associated with the first immune cell and the second immune cell, wherein the comparison criteria is met when the V segments portions have the same length.

Embodiment 16: The method of any of embodiments 1-10, and 15, further including determining a number of base differences between the V segments portions associated with the first immune cell and the second immune cell, and determining that the comparison criteria is not met if the number of base differences exceed a predetermined V base difference threshold.

Embodiment 17: The method of any of embodiments 1-10, 15, and 16, wherein the predetermined V base difference threshold is between about 24 and about 50 bases.

Embodiment 18: The method of any of embodiments any of embodiments 1-10, 15, 16, and 17, wherein the predetermined V base difference threshold is 50 bases.

Embodiment 19: The method of any of embodiments 1-10, the comparison protocol comprising comparing only J segment portions of the immune cell receptor sequences associated with the first immune cell and the second immune cell, wherein the comparison criteria is met when the J segment portions have the same length.

Embodiment 20: The method of any of embodiments 1-10, and 19, further including determining a number of base differences between the J segment portions associated with the first immune cell and the second immune cell, and determining that the comparison criteria is not met if the number of base differences exceed a predetermined J base difference threshold.

Embodiment 21: The method of any of embodiments 1-10, 19, and 20, wherein the predetermined J base difference threshold is between about 24 and about 50 bases.

Embodiment 22: The method of any of embodiments 1-10, 19, 20, and 21, wherein the predetermined J base difference threshold is 50 bases.

Embodiment 23: The method of any of embodiments 1-22, the comparison protocol comprising comparing lengths of the complementarity-determining regions (CDRs) associated with the first immune cell and the second immune cell, and determining that the comparison criteria is met when the CDRs have the same length.

Embodiment 24: The method of any of embodiments 1-23, further comprising determining the number of differences in the CDRs associated with the first immune cell and the second immune cell, and determining that the comparison criteria is not met when the number of differences exceeds a predetermined CDR threshold.

Embodiment 25: The method of any of embodiments 1-24, the comparison protocol comprising determining that the comparison criteria is not met if a first barcode associated with the first immune cell and a second barcode associated with the second immune cell are the same.

Embodiment 26: The method of any of embodiments 1-25, wherein the first immune cell and the second immune cell are cell members of a two-cell clonotype, the method further comprising: determining the number of CDR differences between the cell members; and determining that the comparison criteria is not met if the number of CDR differences exceed a determined two-cell threshold.

Embodiment 27: The method of any of embodiments 1-26, wherein the two-cell threshold has a value dependent on the number of shared mutations.

Embodiment 28: The method of any of embodiments 1-27, further comprising identifying exact subclonotypes within the identified clonotype, wherein the exact subclonotype comprises a non-zero set of immune cells having identical V, D, and J transcripts.

Embodiment 29: The method of any of embodiments 1-28, wherein the exact subclonotype further comprises cells having characteristics selected from the group consisting of a same number of chains, an identical C segment, a same distance between a J stop codon and a C start codon, and combinations thereof.

Embodiment 30: The method of any of embodiments 1-29, wherein the exact subclonotype comprises cells having two or three chains.

Embodiment 31: The method of any of embodiments 1-28, wherein the exact subclonotype comprises cells having a shared inferred antigen specificity, wherein the shared inferred antigen specificity is also shared or not shared by other subclonotypes.

Embodiment 32: The method of any of embodiments 1-28, wherein the exact subclonotype comprises cells having a shared gene expression, surface protein, intracellular protein, nucleotide variant, other cellular features, and combinations thereof.

Embodiment 33: The method of any of embodiments 1-32, wherein the first immune cell and the second immune cell are both B cells.

Embodiment 34: The method of any of embodiments 1-32, wherein the first immune cell and the second immune cell are both T cells.

Embodiment 35: The method of any of embodiments 1-32, wherein the first immune cell and the second immune cell are both dual-expresser (DE) cells, wherein the DE cells express both a T cell receptor and a B cell receptor.

Embodiment 36: The method of any of embodiments 1-32, wherein the first immune cell and the second immune cell are both cells expressing a chimeric antigen receptor.

Embodiment 37: The method of any of embodiments 1-36, wherein the reference immune receptor cell sequence is a donor reference sequence, a universal reference sequence, and combinations thereof.

Embodiment 38: The method of any of embodiments 1-37, wherein the reference immune cell receptor sequence comprises each of the heavy and light chain V segments portions of the immune cell receptor sequences.

Embodiment 39: The method of any of embodiments 1-38, wherein the V segments portion of the immune cell receptor sequences is a B cell heavy chain V segments portion, a B cell light chain V segments portion, a T cell alpha chain V segments portion, a T cell beta chain V segments portion, a T cell gamma chain V segments portion, a T cell delta chain V segments portion, and combinations thereof.

Embodiment 40: The method of any of embodiments 1-39, wherein the donor reference sequence is derived from all the immune cells represented within the immune cell receptor sequence dataset, if there are sufficient immune cells present to determine a high-confidence estimate of the donor reference sequence.

Embodiment 41: The method of any of embodiments 1-40, wherein the plurality of full-length immune cell receptor sequences comprise a variable region immune cell receptor sequence and a constant region immune cell receptor sequence.

Embodiment 42: The method of any of embodiments 1-41, wherein the variable region sequence comprises a V through J segments portion of the immune cell receptor sequence.

Embodiment 43: The method of any of embodiments 1-42, the method comprising determining germline alleles for a donor V segments portion sequence.

Embodiment 44: The method of any of embodiments 1-43, wherein determining the germline alleles for the donor V segments portion sequence comprises calculating shared differences between immune cells with disjoint CDR3 junction sequences within the immune cell receptor sequence dataset and shared mutations relative to a universal reference sequence, and wherein the determination of shared differences are representative of germline mutations.

Embodiment 45: The method of any of embodiments 1-44, the method comprises joining singletons, wherein the singletons can be joined to obtain a more frequent exact subclonotype if the sequences of the singletons are identical.

Embodiment 46: A system for grouping immune cells within an immune cell receptor sequence dataset, the system comprising: a data source configured to obtain the immune cell receptor sequence dataset from a sample, the dataset including a plurality of full-length immune cell receptor sequences each comprised of at least one heavy chain region sequence and one light chain region sequence, wherein each immune cell receptor sequence is associated with an individual immune cell in the sample; and a processing unit configured to receive the immune cell receptor sequence dataset from the data source, the processing unit comprising: a comparison engine configured to compare the immune receptor sequences associated with a first immune cell and a second immune cell from the sample using a comparison protocol, and an identification engine configured to identify the first immune cell and the second immune cell as members of the same clonotype, if one or more immune cell receptor sequence comparison criteria is met.

Embodiment 47: The system of embodiment 46, the comparison engine configured to: receive a reference immune cell receptor sequence; compare the immune cell receptor sequences of the first immune cell and the second immune cell to the reference immune receptor sequence; and determine a number of shared mutations in the immune cell receptor sequences of the first immune cell and the second immune cell.

Embodiment 48: The system of embodiment 46 or 47, wherein the identification engine is further configured to: determine that the comparison criteria is met when the immune cell receptor sequences of the first immune cell and the second immune cell share a pre-set number of mutations.

Embodiment 49: The system of any of embodiments 46-48, the identification engine is further configured to: calculate a probability value that the number of shared mutations occurred by chance; and determine that the comparison criteria is not being met if the probability value exceeds a pre-set probability threshold.

Embodiment 50: The system of any of embodiments 46-49, wherein the pre-set probability threshold is between about 1% and about 0.000001%.

Embodiment 51: The system of any of embodiments 46-50, wherein the pre-set number of shared mutations is 25.

Embodiment 52: The system of any of embodiments 46-51, wherein the pre-set number of shared mutation is 10.

Embodiment 53: The system of any of embodiments 46-52, wherein the shared mutations can be point mutations, chromosomal mutations, or combinations thereof.

Embodiment 54: The system of any of embodiments 46-53, wherein the point mutation is selected from the group consisting of substitution, insertion, and deletion, and the chromosomal mutation is selected from the group consisting inversion, deletion, duplication, translocation, and recombination.

Embodiment 55: The system of any of embodiments 46-54, wherein the point mutation is a somatic hypermutation.

Embodiment 56: The system of any of embodiments 46-55, wherein: the comparison engine is further configured to compare V and J segments portions of the immune cell receptor sequences associated with the first immune cell and the second immune cell, and the identification engine is further configured to determine that the comparison criteria is met when the V and J segments portions have the same length.

Embodiment 57: The system of any of embodiments 46-56, wherein: the comparison engine is further configured to determine the number of base differences between the V and J segments portions associated with the first immune cell and the second immune cell, and the identification engine is further configured to determine that the comparison criteria is not met if the number of base differences exceed a predetermined VJ base difference threshold.

Embodiment 58: The system of any of embodiments 46-57, wherein the predetermined VJ base difference threshold is between about 24 and about 50 bases.

Embodiment 59: The system of any of embodiments 46-58, wherein the predetermined VJ base difference threshold is 50 bases.

Embodiment 60: The system of any of embodiments 46-55, wherein: the comparison engine is further configured to compare only V segments portions of the immune cell receptor sequences associated with the first immune cell and the second immune cell, and the identification engine is further configured to determine that the comparison criteria is met when the V segments portions have the same length.

Embodiment 61: The system of any of embodiments 46-55, and 60, wherein: the comparison engine is configured to determine the number of base differences between the V segments portions associated with the first immune cell and the second immune cell, and the identification engine is configured to determine that the comparison criteria is not met if the number of base differences exceed a predetermined V base difference threshold.

Embodiment 62: The system of any of embodiments 46-55, 60, and 61, wherein the predetermined V base difference threshold is between about 24 and about 50 bases.

Embodiment 63: The system of any of embodiments 46-55, 60, 61, and 62, wherein the predetermined V base difference threshold is 50 bases.

Embodiment 64: The system of any of embodiments 46-55, wherein: the comparison engine is configured to compare only J segment portions of the immune cell receptor sequences associated with the first immune cell and the second immune cell, and the identification engine is configured to determine that the comparison criteria is met when the J segment portions have the same length.

Embodiment 65: The system of any of embodiments 46-55, and 64, wherein: the comparison engine is configured to determine the number of base differences between the J segment portions associated with the first immune cell and the second immune cell, and the identification engine is configured to determine that the comparison criteria is not met if the number of base differences exceed a predetermined J base difference threshold.

Embodiment 66: The system of any of embodiments 46-55, 64, and 65, wherein the predetermined J base difference threshold is between about 24 and about 50 bases.

Embodiment 67: The system of any of embodiments 46-55, 64, 65, and 66, wherein the predetermined J base difference threshold is 50 bases.

Embodiment 68: The system of any of embodiments 46-67, wherein: the comparison engine is configured to compare the lengths of the complementarity-determining regions (CDRs) associated with the first immune cell and the second immune cell, and the identification engine is further configured to determine that the comparison criteria is met when the CDRs have the same length.

Embodiment 69: The system of any of embodiments 46-68, wherein: the comparison engine is configured to determine the number of differences in the CDRs associated with the first immune cell and the second immune cell, and the identification engine is further configured to determine that the comparison criteria is not met when the number of differences exceeds a predetermined CDR threshold.

Embodiment 70: The system of any of embodiments 46-69, wherein: the identification engine is further configured to determine that the comparison criteria is not met if a first barcode associated with the first immune cell and a second barcode associated with the second immune cell are the same.

Embodiment 71: The system of any of embodiments 46-70, wherein the first immune cell and the second immune cell are cell members of a two-cell clonotype, wherein: the comparison engine is further configured to determine the number of CDR differences between the cell members, and the identification engine is further configured to determine that the comparison criteria is not met if the number of CDR differences exceed a determined two-cell threshold.

Embodiment 72: The system of any of embodiments 46-71, wherein the two-cell threshold has a value dependent on the number of shared mutations.

Embodiment 73: The system of any of embodiments 46-72, wherein the identification engine is configured to identify exact subclonotypes within the identified clonotype, wherein the exact subclonotype comprises a non-zero set of immune cells having identical V, D, and J transcripts.

Embodiment 74: The system of any of embodiments 46-73, wherein the exact subclonotype further comprises cells having characteristics selected from the group consisting of a same number of chains, an identical C segment, a same distance between a J stop codon and a C start codon, and combinations thereof.

Embodiment 75: The system of any of embodiments 46-74, wherein the exact subclonotype comprises cells having two or three chains.

Embodiment 76: The system of any of embodiments 46-73, wherein the exact subclonotype comprises cells having a shared inferred antigen specificity, wherein the shared inferred antigen specificity is also shared or not shared by other subclonotypes.

Embodiment 77: The system of any of embodiments 46-73, wherein the exact subclonotype comprises cells having a shared gene expression, surface protein, intracellular protein, nucleotide variant, other cellular features, and combinations thereof.

Embodiment 78: The system of any of embodiments 46-77, wherein the first immune cell and the second immune cell are both B cells.

Embodiment 79: The system of any of embodiments 46-77, wherein the first immune cell and the second immune cell are both T cells.

Embodiment 80: The system of any of embodiments 46-77, wherein the first immune cell and the second immune cell are both dual-expresser (DE) cells, wherein the DE cells express both a T cell receptor and a B cell receptor.

Embodiment 81: The system of any of embodiments 46-77, wherein the first immune cell and the second immune cell are both cells expressing a chimeric antigen receptor.

Embodiment 82: The system of any of embodiments 46-81, wherein the reference immune receptor cell sequence is a donor reference sequence, a universal reference sequence, and combinations thereof.

Embodiment 83: The system of any of embodiments 46-82, wherein the reference immune cell receptor sequence comprises each of the heavy and light chain V segments portions of the immune cell receptor sequences.

Embodiment 84: The system of any of embodiments 46-83, wherein the V segments portion of the immune cell receptor sequences is a B cell heavy chain V segments portion, a B cell light chain V segments portion, a T cell alpha chain V segments portion, a T cell beta chain V segments portion, a T cell gamma chain V segments portion, a T cell delta chain V segments portion, and combinations thereof.

Embodiment 85: The system of any of embodiments 46-84, wherein the donor reference sequence is derived from all the immune cells represented within the immune cell receptor sequence dataset, if there are sufficient immune cells present to determine a high-confidence estimate of the donor reference sequence.

Embodiment 86: The system of any of embodiments 46-85, wherein the plurality of full-length immune cell receptor sequences comprise a variable region immune cell receptor sequence and a constant region immune cell receptor sequence.

Embodiment 87: The system of any of embodiments 46-86, wherein the variable region sequence comprises a V through J segments portion of the immune cell receptor sequence.

Embodiment 88: The system of any of embodiments 46-87, wherein: the identification engine is further configured to determine germline alleles for a donor V segments portion sequence.

Embodiment 89: The system of any of embodiments 46-88, wherein: the comparison engine is further configured to determine shared differences between immune cells within the immune cell receptor sequence dataset with disjoint CDR3 junction sequences and shared differences relative to a universal reference sequence, and the identification engine is further configured to determine that the identification of the shared differences are representative of germline mutations.

Embodiment 90: The system of any of embodiments 46-89, wherein: the identification engine is further configured to join singletons to obtain a more frequent exact subclonotype if the sequences of the singletons are identical.

Embodiment 91: A non-transitory computer-readable medium in which a program is stored for causing a computer to perform a method for grouping immune cells within an immune cell receptor sequence dataset, the method comprising: obtaining the immune cell immune receptor sequence dataset from a sample, the dataset including a plurality of full-length immune cell receptor sequences each comprised of at least one heavy chain region sequence and one light chain region sequence, wherein each immune cell receptor sequence is associated with an individual immune cell in the sample; comparing the immune cell receptor sequences associated with a first immune cell and a second immune cell from the sample using a comparison protocol; and identifying the first immune cell and the second immune cell as members of the same clonotype, if one or more immune cell receptor sequence comparison criteria is met.

Embodiment 92: The method of embodiment 91, further including: receiving a reference immune cell receptor sequence; comparing the immune receptor sequences of the first immune cell and the second immune cell to the reference immune cell receptor sequence; and determining a number of shared mutations in the immune cell receptor sequences of the first immune cell and the second immune cell.

Embodiment 93: The method of embodiment 91 or 92, wherein the comparison criteria is met when the immune receptor sequences of the first immune cell and the second immune cell share a pre-set number of mutations.

Embodiment 94: The method of any of embodiments 91-93, further including: calculating a probability value that the number of shared mutations occurred by chance; and determining the comparison criteria as not being met if the probability value exceeds a pre-set probability threshold.

Embodiment 95: The method of any of embodiments 91-94, wherein the pre-set probability threshold is between about 1% and about 0.000001%.

Embodiment 96: The method of any of embodiments 91-95, wherein the pre-set number of shared mutations is 25.

Embodiment 97: The method of any of embodiments 91-96, wherein the pre-set number of shared mutation is 10.

Embodiment 98: The method of any of embodiments 91-97, wherein the shared mutations can be point mutations, chromosomal mutations, or combinations thereof.

Embodiment 99: The method of any of embodiments 91-98, wherein the point mutation is selected from the group consisting of substitution, insertion, and deletion, and the chromosomal mutation is selected from the group consisting inversion, deletion, duplication, translocation, and recombination.

Embodiment 100: The method of any of embodiments 91-99, wherein the point mutation is a somatic hypermutation.

Embodiment 101: The method of any of embodiments 91-100, wherein V and J segments portions of the immune cell receptor sequences associated with the first immune cell and the second immune cell are compared, wherein the comparison criteria is met when the V and J segments portions have the same length.

Embodiment 102: The method of any of embodiments 91-101, further including determining a number of base differences between the V and J segment portions associated with the first immune cell and the second immune cell, and determining that the comparison criteria is not met if the number of base differences exceed a predetermined VJ base difference threshold.

Embodiment 103: The method of any of embodiments 91-102, wherein the predetermined VJ base difference threshold is between about 24 and about 50 bases.

Embodiment 104: The method of any of embodiments 91-103, wherein the predetermined VJ base difference threshold is 50 bases.

Embodiment 105: The method of any of embodiments 91-100, wherein only V segments portions of the immune cell receptor sequences associated with the first immune cell and the second immune cell are compared, wherein the comparison criteria is met when the V segments portions have the same length.

Embodiment 106: The method of any of embodiments 91-100, and 105, further including determining a number of base differences between the V segments portions associated with the first immune cell and the second immune cell, and determining that the comparison criteria is not met if the number of base differences exceed a predetermined V base difference threshold.

Embodiment 107: The method of any of embodiments 91-100, 105, and 106, wherein the predetermined V base difference threshold is between about 24 and about 50 bases.

Embodiment 108: The method of any of embodiments 91-100, 105, 106, and 107, wherein the predetermined V base difference threshold is 50 bases.

Embodiment 109: The method of any of embodiments 91-100, wherein only J segment portions of the immune cell receptor sequences associated with the first immune cell and the second immune cell are compared, wherein the comparison criteria is met when the J segment portions have the same length.

Embodiment 110: The method of any of embodiments 91-100, and 109, further including determining a number of base differences between the J segment portions associated with the first immune cell and the second immune cell, and determining that the comparison criteria is not met if the number of base differences exceed a predetermined J base difference threshold.

Embodiment 111: The method of any of embodiments 91-100, 109, and 110, wherein the predetermined J base difference threshold is between about 24 and about 50 bases.

Embodiment 112: The method of any of embodiments 91-100, 109, 110, and 111, wherein the predetermined J base difference threshold is 50 bases.

Embodiment 113: The method of any of embodiments 91-112, further comprising comparing lengths of the complementarity-determining regions (CDRs) associated with the first immune cell and the second immune cell, and determining that the comparison criteria is met when the CDRs have the same length.

Embodiment 114: The method of any of embodiments 91-113, further comprising determining the number of differences in the CDRs associated with the first immune cell and the second immune cell, and determining that the comparison criteria is not met when the number of differences exceeds a predetermined CDR threshold.

Embodiment 115: The method of any of embodiments 91-114, further comprising determining that the comparison criteria is not met if a first barcode associated with the first immune cell and a second barcode associated with the second immune cell are the same.

Embodiment 116: The method of any of embodiments 91-115, wherein the first immune cell and the second immune cell are cell members of a two-cell clonotype, the method further comprising: determining the number of CDR differences between the cell members; and determining that the comparison criteria is not met if the number of CDR differences exceed a determined two-cell threshold.

Embodiment 117: The method of any of embodiments 91-116, wherein the two-cell threshold has a value dependent on the number of shared mutations.

Embodiment 118: The method of any of embodiments 91-117, further comprising identifying exact subclonotypes within the identified clonotype, wherein the subclonotype comprises a non-zero set of immune cells having identical V, D, and J transcripts.

Embodiment 119: The method of any of embodiments 91-118, wherein the exact subclonotype further comprises immune cells having characteristics selected from the group consisting of a same number of chains, an identical C segment, a same distance between a J stop codon and a C start codon, and combinations thereof.

Embodiment 120: The method of any of embodiments 91-119, wherein the subclonotype comprises cells having two or three chains.

Embodiment 121: The method of any of embodiments 91-118, wherein the exact subclonotype comprises cells having a shared inferred antigen specificity, wherein the shared inferred antigen specificity is also shared or not shared by other subclonotypes.

Embodiment 122: The method of any of embodiments 91-118, wherein the exact subclonotype comprises cells having a shared gene expression, surface protein, intracellular protein, nucleotide variant, other cellular features, and combinations thereof.

Embodiment 123: The method of any of embodiments 91-122, wherein the first immune cell and the second immune cell are both B cells.

Embodiment 124: The method of any of embodiments 91-122, wherein the first immune cell and the second immune cell are both T cells.

Embodiment 125: The method of any of embodiments 91-122, wherein the first immune cell and the second immune cell are both dual-expresser (DE) cells, wherein the DE cells express both a T cell receptor and a B cell receptor.

Embodiment 126: The method of any of embodiments 91-122, wherein the first immune cell and the second immune cell are both cells expressing a chimeric antigen receptor.

Embodiment 127: The method of any of embodiments 91-116, wherein the reference immune receptor cell sequence is a donor reference sequence, a universal reference sequence, and combinations thereof.

Embodiment 128: The method of any of embodiments 91-117, wherein the reference immune cell receptor sequence comprises each of the heavy and light chain V segments portions of the immune cell receptor sequences.

Embodiment 129: The method of any of embodiments 91-118, wherein the V segments portion of the immune cell receptor sequences is a B cell heavy chain V segments portion, a B cell light chain V segments portion, a T cell alpha chain V segments portion, a T cell beta chain V segments portion, a T cell gamma chain V segments portion, a T cell delta chain V segments portion, and combinations thereof.

Embodiment 130: The method of any of embodiments 91-119, wherein the donor reference sequence is derived from all the immune cells represented within the immune cell receptor sequence dataset, if there are sufficient immune cells present to determine a high-confidence estimate of the donor reference sequence.

Embodiment 131: The method of any of embodiments 91-120, wherein the plurality of full-length immune cell receptor sequences comprise a variable region immune cell receptor sequence and a constant region immune cell receptor sequence.

Embodiment 132: The method of any of embodiments 91-121, wherein the variable region sequence comprises a V through J segments portion of the immune cell receptor sequence.

Embodiment 133: The method of any of embodiments 91-122, the method comprises determining the germline alleles for a donor V segments portion sequence.

Embodiment 134: The method of any of embodiments 91-123, wherein determining the germline alleles for a donor V segments portion sequence comprises calculating shared differences between immune cells with disjoint CDR3 junction sequences within the immune cell receptor sequence dataset and shared mutations relative to a universal reference sequence, and wherein the determination of shared differences are representative of germline mutations.

Embodiment 135: The method of any of embodiments 91-124, the method comprises joining singletons, wherein the singletons can be joined to obtain a more frequent exact subclonotype if the sequences of the singletons are identical.

Embodiment 136: The method of embodiment 1, the comparison protocol comprising comparing D segments portions of the immune cell receptor sequences associated with the first immune cell and the second immune cell, wherein the comparison criteria is met when the D segments portions have the same length.

Embodiment 137: The system of embodiment 46, wherein: the comparison engine is further configured to compare D segments portions of the immune cell receptor sequences associated with the first immune cell and the second immune cell, and the identification engine is further configured to determine that the comparison criteria is met when the D segments portions have the same length.

Embodiment 138: The method of embodiment 91, wherein D segments portions of the immune cell receptor sequences associated with the first immune cell and the second immune cell are compared, wherein the comparison criteria is met when the D segments portions have the same length.

1. A method for grouping immune cells within an immune cell receptor sequence dataset, the method comprising:
obtaining the immune cell receptor sequence dataset from a sample, the dataset including a plurality of full-length immune cell receptor sequences each comprised of at least one heavy chain region sequence and one light chain region sequence, wherein each immune cell receptor sequence is associated with an individual immune cell in the sample;
comparing the immune cell receptor sequences associated with a first immune cell and a second immune cell from the sample using a comparison protocol; and
identifying the first immune cell and the second immune cell as members of the same clonotype, if one or more immune cell receptor sequence comparison criteria is met.

2. The method of claim 1, the comparison protocol including:
receiving a reference immune cell receptor sequence;
comparing the immune cell receptor sequences of the first immune cell and the second immune cell to the reference immune cell receptor sequence; and
determining a number of shared mutations in the immune cell receptor sequences of the first immune cell and the second immune cell.

3. The method of claim 2, wherein the comparison criteria is met when the immune cell receptor sequences of the first immune cell and the second immune cell share a pre-set number of mutations.

4. The method of claim 3, the comparison protocol including:
calculating a probability value that the number of shared mutations occurred by chance; and
determining the comparison criteria as not being met if the probability value exceeds a pre-set probability threshold.

5. The method of claim 1, the comparison protocol comprising comparing V and J segments portions of the immune cell receptor sequences associated with the first immune cell and the second immune cell, wherein the comparison criteria is met when the V and J segments portions have the same length.

6. The method of claim 5, further including determining a number of base differences between the V and J segments portions associated with the first immune cell and the second immune cell, and determining that the comparison criteria is not met if the number of base differences exceed a predetermined VJ base difference threshold.

7. The method of claim 1, the comparison protocol comprising comparing lengths of the complementarity-determining regions (CDRs) associated with the first immune cell and the second immune cell, and determining that the comparison criteria is met when the CDRs have the same length.

8. The method of claim 7, further comprising determining the number of differences in the CDRs associated with the first immune cell and the second immune cell, and determining that the comparison criteria is not met when the number of differences exceeds a predetermined CDR threshold.

9. The method of claim 1, wherein the first immune cell and the second immune cell are cell members of a two-cell clonotype, the method further comprising:
determining the number of CDR differences between the cell members;
determining that the comparison criteria is not met if the number of CDR differences exceed a determined two-cell threshold.

10. The method of claim 9, wherein the two-cell threshold has a value dependent on the number of shared mutations.

11. The method of claim 1, further comprising identifying exact subclonotypes within the identified clonotype, wherein the exact subclonotype comprises a non-zero set of immune cells having identical V, D, and J transcripts.

12. The method of claim 1, the method comprising determining germline alleles for a donor V segments portion sequence.

13. The method of claim 12, wherein determining the germline alleles for the donor V segments portion sequence comprises calculating shared differences between immune cells with disjoint CDR3 junction sequences within the immune cell receptor sequence dataset and shared mutations relative to a universal reference sequence, and wherein the determination of shared differences are representative of germline mutations.

14. A system for grouping immune cells within an immune cell receptor sequence dataset, the system comprising
a data source configured to obtain the immune cell receptor sequence dataset from a sample, the dataset including a plurality of full-length immune cell receptor sequences each comprised of at least one heavy chain region sequence and one light chain region sequence, wherein each immune cell receptor sequence is associated with an individual immune cell in the sample; and
a processing unit configured to receive the immune cell receptor sequence dataset from the data source, the processing unit comprising:
a comparison engine configured to compare the immune receptor sequences associated with a first immune cell and a second immune cell from the sample using a comparison protocol, and
an identification engine configured to identify the first immune cell and the second immune cell as members of the same clonotype, if one or more immune cell receptor sequence comparison criteria is met.

15. The system of claim 14, the comparison engine configured to:
receive a reference immune cell receptor sequence;
compare the immune cell receptor sequences of the first immune cell and the second immune cell to the reference immune receptor sequence; and
determine a number of shared mutations in the immune cell receptor sequences of the first immune cell and the second immune cell.

16.-26. (canceled)

27. A non-transitory computer-readable medium in which a program is stored for causing a computer to perform a method for grouping immune cells within an immune cell receptor sequence dataset, the method comprising:
obtaining the immune cell immune receptor sequence dataset from a sample, the dataset including a plurality of full-length immune cell receptor sequences each comprised of at least one heavy chain region sequence and one light chain region sequence, wherein each immune cell receptor sequence is associated with an individual immune cell in the sample;
comparing the immune cell receptor sequences associated with a first immune cell and a second immune cell from the sample using a comparison protocol; and
identifying the first immune cell and the second immune cell as members of the same clonotype, if one or more immune cell receptor sequence comparison criteria is met.

28. The non-transitory computer-readable medium of claim 27, further including:
receiving a reference immune cell receptor sequence;
comparing the immune receptor sequences of the first immune cell and the second immune cell to the reference immune cell receptor sequence; and
determining a number of shared mutations in the immune cell receptor sequences of the first immune cell and the second immune cell.

29.-42. (canceled)

43. The method of claim 1, the method comprises joining singletons, wherein the singletons can be joined to obtain a more frequent exact subclonotype if the sequences of the singletons are identical.

44. The method of claim 1, the comparison protocol comprising comparing D segments portions of the immune cell receptor sequences associated with the first immune cell and the second immune cell, wherein the comparison criteria is met when the D segments portions have the same length.

45.-46. (canceled)
